(19) United States
(12) Patent Application Publication
(10) Pub. No.: US 2021/0118558 A1
(43) Pub. Date: Apr. 22, 2021

(54) SYSTEM AND METHOD FOR IMPROVING HEALTHCARE THROUGH COMMUNITY ENGAGEMENT

(71) Applicant: Cleveland State University, Cleveland, OH (US)

(72) Inventor: William A. MATCHAM, Elyria, OH (US)

(73) Assignee: Cleveland State University, Cleveland, OH (US)

(21) Appl. No.: 17/072,984

(22) Filed: Oct. 16, 2020

Related U.S. Application Data
(60) Provisional application No. 62/916,610, filed on Oct. 17, 2019.

(51) Int. Cl. G16H 40/20; G16H 10/20; G16H 50/70; G16H 10/40; G16H 50/80; G16H 80/00; G06Q 10/10; G16H 10/60; G06F 21/62; G06F 16/9535; A61B 5/00; G06F 3/0482; H04L 29/06

(52) U.S. Cl. CPC G16H 40/20; G16H 10/20; G16H 50/70; G16H 10/40; G16H 50/80; G16H 80/00; H04L 67/42; G16H 10/60; G06F 21/6254; G06F 16/9535; A61B 5/743; A61B 5/7475; G06F 3/0482; G06Q 10/1097

(57) ABSTRACT

Methods and systems for improving healthcare through community engagement are provided. In one embodiment, a method includes initializing a community health application on a computing device for an engagement session with a candidate patient. A client identification sequence is performed using the application to enroll the candidate patient as a new community health client or lookup the candidate patient as an existing community health client. Certain client information is collected from the community health client via interaction with the computing device by a healthcare facilitator during the engagement session. The community health application controlling the computing device by presenting displays and reading data in relation to the displays. The client information is processed to select a clinical sequence for the engagement session with the community health client based on the client information. In another embodiment, the method is directed to processing and distribution of the client information to third parties.

### CROSS-REFERENCE TO RELATED APPLICATION

This application claims priority to and the benefit of U.S. Provisional Patent Application Ser. No. 62/916,610, filed Oct. 17, 2019 and entitled SYSTEM AND METHOD FOR IMPROVING HEALTHCARE THROUGH COMMUNITY ENGAGEMENT, the contents of which are fully incorporated herein by reference.

### BACKGROUND

This disclosure presents various embodiments of a system and method for improving healthcare through community engagement. In one embodiment, the system and method are implemented using a community health application on a mobile or portable computing device to facilitate collection of information from a client during an engagement session between the client and a healthcare facilitator. In other embodiments, the system and method are implemented using a server computing system to store information collected from a client during an engagement session between the client and a healthcare facilitator and process the information for various specialized purposes, including de-identifying the information to form anonymous community health information.

The healthcare system in the United States continues to go through transformation to address escalating costs that have limited the public's ability to access affordable, high-quality health and medical care. This results in the need for approaches to provide increased access to primary care. For example, approaches that will help hospitals and health systems to decrease readmissions and emergency department (ED) visits, increase patient adherence, improve health and wellness, reduce risk, prevent disease, and meet population needs identified by community health needs assessments (CHNAs).

One such approach is the implementation of a community health worker (CHW) program. A growing body of evidence points to the positive health impacts by CHWs who address the needs of individuals facing barriers to healthcare access due to cultural practices, race, ethnicity, language, literacy, geography, income, ability, or other related factors. In coordination with mainstream healthcare providers, CHWs offer health, wellness, and disease prevention and management services to decrease health disparities and achieve improved health outcomes, better patient experience, and increased affordability.

The American Public Health Association defines CHWs as “a frontline public health worker who is a trusted member and/or has a close understanding of the community served.” The Ohio Board of Nursing defines CHWs as: “a community representative who advocates for clients in the community by assisting them in accessing community health and supportive resources through the provision of such services as education, role modeling, outreach, home visits, or referrals.” The actual working title of a CHW, especially an uncertified CHW, varies across a given jurisdictional boundary. For example, a person performing tasks assigned to CHWs may go by other titles, such as community health representative, community health advisor, health coach, family advocate, health educator, liaison, outreach worker, patient navigator, public health aide, patient care coordinator, and promotores de salud.

Medically underserved patients do not see doctors on a regular basis, if at all. A CHW program can focus on preventative healthcare in the community to avoid hospital admissions. Traditionally, medically underserved individuals will receive increased access to services under a CHW program. For example, services that can improve factors related to social determinants of health may address lack of healthy food choices, affordable transportation, insecure housing, environmental factors, crime, and insufficient recreational opportunities.

Healthcare delivery systems can benefit from the skills, community knowledge and cultural competency that CHWs possess to connect with those at risk for poor health outcomes. For example, CHW interventions can significantly decrease HbA1c (see, e.g., Perez-Escamilla, et al., Diabetes Care 38, 197-205, 2015). Similarly, CHW interventions can reduce asthma triggers which can lead to reduced ED and/or urgent care (UC) visits (see, e.g., Postma, et al., J Asthma 46 (6) 564-576, 2009). Public and private payers may see reductions in total healthcare spending (e.g., reduced hospital admissions) where a CHW program is implemented.

In Ohio, the State Board of Nursing offers certification and regulates CHW practice. Currently, there are about 850 certified and non-certified CHWs in Ohio. Currently, Ohio organizations can hire un-certified CHWs, but state laws will soon require all CHWs to be certified. As of Sep. 9, 2020, there are only eighteen Ohio Board of Nursing (OBN) approved CHW training programs and one provisionally approved CHW training program in Ohio.

For example, in Ohio, the following organizations employ CHWs: MetroHealth Medical Center, University Hospitals, Neighborhood Family Practice, Northeast Ohio Neighborhood (NEON) Health Services, Care Source (e.g., public programs such as Medicaid, Medicare, and Marketplace), Mom's First, and community agencies (e.g., Friendly Inn Settlement House, health departments, and federally-qualified health centers). Care Source is the largest Medicaid plan in Ohio and second largest Medicaid plan in the United States.

There are currently no applications for CHWs to collect client information using mobile or portable computing devices at remote or temporary facilities, such as the client's home, local church, employer facility, local government facility, or other public or private facilities. Data collection by CHW at these facilities is typically done by hand with paper and pencil. Modern electronic medical record (EMR)/electronic health record (EHR) systems do not have community data collection capabilities that can be used by CHWs. Under these circumstances, labor-intensive data entry services may be required to integrate the client information collected by CHWs into EMR/EHR systems.

The problems created by the ‘paper and clipboard’ recording systems used by CHWs include: 1) disparate systems of data collection and compliance, 2) inefficient methods of data collection, 3) data entry errors and risks of duplication, 4) difficulty in reporting out of this system, 5) loss of information due to misplacement/damage to paper copy, 6) no reasonable way to share historical patient records across different visits/clinics, 7) lack of referral mechanisms and follow-up, and 8) lack of a clinical decision support system (CDSS) to recommend education/interventions based on best practice guidelines. For example, current CHW programs may rely on the individual level of knowledge of CHWs which is highly variable.

Currently, the client data collected by CHWs via pen and paper may be given to an agency sponsoring an event reaching out to the community. The client data collected at ‘pop up’ events may also be given to the client. Thus, there are needs for techniques to electronically collect community health data, process the data, retain the data, distribute the data, and integrate the data into EMR/EHR systems.

### BRIEF SUMMARY

In one aspect, a method for improving healthcare through community engagement is provided. In one embodiment, the method includes: initializing a community health application on a computing device for an engagement session with a candidate patient from a community; performing a client identification sequence using the community health application to enroll the candidate patient as a new community health client or lookup the candidate patient as an existing community health client; collecting first client information from the community health client via interaction with a user interface of the computing device by a healthcare facilitator during the engagement session, the community health application controlling the user interlace by presenting displays on a display device and reading data from the user interface in relation to the displays; and processing the first client information to select a clinical sequence for the engagement session with the community health client based at least in part on the first client information.

In another aspect, another method for improving healthcare through community engagement is provided. In one embodiment, the method includes: storing client information from engagement sessions with a plurality of community health clients in a centralized community health information database of a server computing system, the client information from the engagement sessions being captured electronically during the engagement sessions by healthcare facilitators using a community health application on computing devices, each engagement session including a client identification sequence controlled by the community health application to identify the corresponding community health client, collect the client information from the community health client, and select a clinical sequence for the engagement session based on the client information; providing an authorized person with access to the client information in the centralized community health information database via a remote computing device with access to the server computing system; processing a database query from the remote computing device at the server computing system; and providing results of the database query from the server computing system to the remote computing device.

In yet another aspect, a computerized system for improving healthcare through community engagement is provided. In one embodiment, the computerized system includes a computing device configured for use by a healthcare facilitator for an engagement session with a candidate patient from a community. The computing device including at least one processor and associated memory, a user interface, a display device, and at least one storage device. The at least one processor, associated memory, user interface, display device, and at least one storage device are configured to initialize a community health application on the computing device for the engagement session. The at least one processor, associated memory, user interface, display device, and at least one storage device are configured to perform a client identification sequence using the community health application to enroll the candidate patient as a new community health client or lookup the candidate patient as an existing community health client. The at least one processor, associated memory, user interface, display device, and at least one storage device are configured to collect first client information from the community health client via interaction with the user interface of the computing device by the healthcare facilitator during the engagement session, the community health application controlling the user interface by presenting displays on the display device and reading data from the user interface in relation to the displays. The at least one processor, associated memory, and at least one storage device are configured to process the first client information to select a clinical sequence for the engagement session with the community health client based at least in part on the first client information.

In still another aspect, another computerized system for improving healthcare through community engagement is provided. In one embodiment, the computerized system includes a server computing system. The server computing system including at least one processor and associated memory, at least one storage device configured to store a centralized community health information database, and at least one network interface device. The at least one processor and associated memory and the at least one storage device are configured to store client information from engagement sessions with a plurality of community health clients in the centralized community health information database. The client information from the engagement sessions being captured electronically during the engagement sessions by healthcare facilitators using a community health application on computing devices. Each engagement session including a client identification sequence controlled by the community health application to identify the corresponding community health client, collect the client information from the community health client, and select a clinical sequence for the engagement session based on the client information. The computing devices being configured to transmit the client information from the engagement sessions to the server computing system and the server computing system being configured to receive the client information via the at least one network interface device. The at least one processor and associated memory, the at least one storage device, and the at least one network interface device are configured to provide an authorized person with access to the client information in the centralized community health information database via a remote computing device. The at least one processor and associated memory and the at least one storage device are configured to process a database query from the remote computing device at the server computing system. The at least one processor and associated memory, the at least one storage device, and the at least one network interface device are configured to provide results of the database query from the server computing system to the remote computing device.

### BRIEF DESCRIPTION OF THE DRAWINGS

FIG. 1 is a flow chart of an exemplary process for improving healthcare through community engagement;

FIG. 2, in combination with FIG. 1, is a flow chart of another exemplary process for improving healthcare through community engagement;

FIG. 3, in combination with FIG. 1, is a flow chart of yet another exemplary process for improving healthcare through community engagement;

FIG. 4, in combination with FIGS. 1 and 3, is a flow chart of still another exemplary process for improving healthcare through community engagement;

FIG. 5, in combination with FIGS. 1, 3, and 4, is a flow chart of still yet another exemplary process for improving healthcare through community engagement;

FIG. 6, in combination with FIGS. 1 and 3, is a flow chart of another exemplary process for improving healthcare through community engagement;

FIG. 7, in combination with FIGS. 1 and 3, is a flow chart of yet another exemplary process for improving healthcare through community engagement;

FIG. 8, in combination with FIGS. 1 and 3, is a flow chart of still another exemplary process for improving healthcare through community engagement;

FIG. 9 is a flow chart of still yet another exemplary process for improving healthcare through community engagement;

FIG. 10, in combination with FIG. 9, is a flow chart of another exemplary process for improving healthcare through community engagement;

FIG. 11, in combination with FIG. 9, is a flow chart of yet another exemplary process for improving healthcare through community engagement;

FIG. 12, in combination with FIGS. 9 and 11, is a flow chart of still another exemplary process for improving healthcare through community engagement;

FIG. 13 is a block diagram of an exemplary system for improving healthcare through community engagement;

FIG. 14 is a block diagram of another exemplary system for improving healthcare through community engagement;

FIGS. 15 through 20 are flow charts of exemplary processes for an exemplary client identification sequence in an engagement session with a candidate patient from a community;

FIGS. 21 through 24 are flow charts of exemplary processes for an exemplary clinical sequence in an engagement session with a community health client;

FIG. 25 is a flow chart of exemplary facilitator intervention and facilitator referral sequences in an engagement session with a community health client;

FIG. 26 is a flow chart of an exemplary process for an exemplary supplemental clinical sequence in an engagement session with a community health client;

FIGS. 27A-B shows exemplary dashboard and clinic displays presented on a computing device by an exemplary community health application;

FIG. 28 shows an exemplary display for a demographics tab of a current assessment presented on a computing device by an exemplary community health application;

FIG. 29 shows an exemplary display for a vitals tab of an exemplary foot clinic presented on a computing device by an exemplary community health application;

FIG. 30 shows an exemplary display for a medical history tab of an exemplary foot clinic presented on a computing device by an exemplary community health application;

FIGS. 31A-B shows exemplary displays for a foot assessment tab of an exemplary foot clinic presented on a computing device by an exemplary community health application;

FIG. 32 shows an exemplary display for an education tab of an exemplary foot clinic presented on a computing device by an exemplary community health application;

FIGS. 33A-B shows exemplary displays for a foot activity tab of an exemplary foot clinic presented on a computing device by an exemplary community health application;

FIG. 34 shows an exemplary display for a referrals tab of an exemplary foot clinic presented on a computing device by an exemplary community health application;

FIG. 35A-E shows an exemplary report summary generated by an exemplary community health application on a computing device for an exemplary foot clinic; and

FIG. 36 is a block diagram of yet another exemplary system for improving healthcare through community engagement.

### DETAILED DESCRIPTION

This disclosure provides various embodiments of systems and methods for improving healthcare through community engagement. For example, embodiments of systems and methods are provided through a community health application on a mobile or portable computing device to facilitate collection of information from a client during an engagement session between the client and a healthcare facilitator. Embodiments of systems and methods are also provided using a server computing system to store information collected from a client during an engagement session between the client and a healthcare facilitator and process the information for various specialized purposes, including de-identifying the information to form anonymous community health information.

In one embodiment of a system and method for improving healthcare through community engagement, mobile application software in a portable computing device facilitates collection of client physiological information, collection of DNA samples, interventions, and education by CHW. For example, a foot clinic workflow can be implemented by the CHW using the mobile application. The mobile application includes several pages of data entry and provides space for Client demographic information, vital signs, medical history, foot care observations and documentation of foot condition and CHW interventions. Both interactive foot models and photographic documentation of feet are possible. The mobile application generates suggestions for educational interventions and referrals to community and healthcare resources. The mobile application generates flags when critical values are out of normal range on vital signs and lab values. Other embodiments can collect data on chronic disease management, environmental health, neonatal/other-baby health, community health clinic, community nutrition, and community athletics.

In various embodiments, the system and method for improving healthcare through community engagement may be referred to as I-Hope (i.e., Improved Health Outcomes via Population Engagement). For example, the I-Hope mobile application tracks and records the activities of CHWs in the community. In one embodiment, the mobile application runs on an iPad. In other embodiments, the mobile application runs on other types of mobile and/or portable computing devices. The mobile application collects population and intervention data in community settings. For example, the mobile application collects data within the scope of practice of the CHW.

In particular, the system and method for improving healthcare through community engagement facilitates collection of data from clients in the community who do not access, or have limited access, to available healthcare systems. The I-Hope application can be implemented on a mobile or portable computing device, conforms to the CHW scope of practice, and captures data from populations that is hard to capture and quantify.

The mobile application can be used in the classroom training of CHWs. The mobile application also has healthcare corporation applicability to fill the data gap between community populations and existing healthcare systems.

Potential end-users for the application are CHWs, their employers, and their clients. The community data gathered by the I-Hope system would be valuable data for use by health departments, universities, nursing, hospitals, and anyone concentrating interventions on chronic disease in the community.

Some of the advantages of the I-Hope (i.e., Improved Health Outcomes via Population Engagement) include: i) removing or reducing paper from the data collection process; ii) using a computerized dashboard to select any of various clinics; iii) using computerized selection of multiple activities; iv) improving efficiency through use of simplified computerized processes; v) using a computerized system to record and retain information; vi) synchronization of disparate data across multiple CHW clinics; v) ability to be integrated into existing EMRs/EHRs; and vi) providing a CDSS that can recommend individualized education and other interventions as well as referrals for I-Hope patients.

The various clinics that could be implemented via the I-Hope system include: i) foot and wound care; ii) dental care; iii) chronic disease management; iv) diabetes; v) cardiovascular (CV) disease, hypertension, and heart failure (HF); vi) pulmonary disease, asthma, and chronic obstructive pulmonary disease (COPD); vii) renal disease and dialysis; viii) mental health and substance abuse; ix) obesity; x) environmental health; xi) lead exposure, radon, mold, and asbestos; xii) nutrition education; xiii) community preventative health fairs; xiv) women's health; xv) pre-natal and baby care (mother-baby), and xvi) sports medicine.

For example, sports medicine may focus on team specific information. The foot clinic can record physiologic observations of feet and record source data problems with feet. The foot clinic can capture CHW specific tasks not recorded in normal visits. The foot clinic can capture images with camera of feet for wound documentation.

The I-Hope system can implement various electronic data capture strategies or can be made compatible with such strategies. For example, data capture strategies such as Medidata Solutions, Veeva Systems, Eversana, IBM Watson, Amazon, Oracle, Qlik, BioClinica, and Clarivate Analytics.

The I-Hope system can implement various monetization strategies. For example, registration and subscription fees for CHW employers and/or hospitals. Accumulated client data can be de-identified and sold. Localized advertising campaigns can be implemented to provide geolocation-based advertising.

Certain features of the I-Hope system differentiate it from existing healthcare practices. For example, the I-Hope mobile application provides a digital data entry system specifically designed to meet the CHW scope of practice. The I-Hope system is independent of proprietary EHR/EMR systems, but data can be shared with such EHR/EMR systems. The I-Hope system has flags built in to notify CHW when to refer a patient to a licensed healthcare provider. The I-Hope system captures healthcare information and facilitates referrals and education in the community that is hard to quantify in traditional EMR systems. The I-Hope system could avoid the need for a healthcare provider license to simply allow CHW access EMR systems.

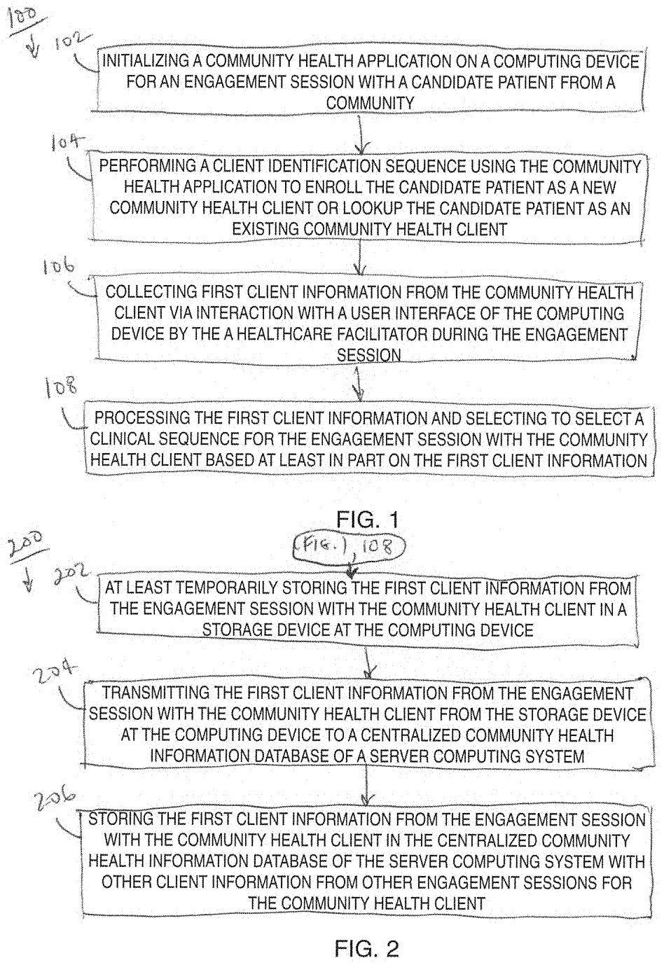

With reference to FIG. 1, an exemplary embodiment of a process 100 for improving healthcare through community engagement begins at 102 where a community health application is initialized on a computing device for an engagement session with a candidate patient from a community. For example, the initializing may include performing a login sequence to allow a healthcare facilitator to run the community health application on the computing device. At 104, a client identification sequence is performed using the community health application to enroll the candidate patient as a new community health client or lookup the candidate patient as an existing community health client. At 106, first client information is collected from the community health client via interaction with a user interface of the computing device by the healthcare facilitator during the engagement session. The community health application controlling the user interface by presenting displays on a display device and reading data from the user interface in relation to the displays. At 108, the first client information is processed, to select a clinical sequence for the engagement session with the community health client based at least in part on the first client information.

In another embodiment of the process 100, the community health application is a web-based application accessible to the computing device via a web browser. In a further embodiment, the web-based application is on the computing device. In another further embodiment, the web-based application is on a server computing system accessible to the computing device.

In yet another embodiment of the process 100, the community health application is a computer-based application on the computing device. In still another embodiment of the process 100, the engagement session includes a face-to-face meeting between the healthcare facilitator and the candidate patient, a phone call between the healthcare facilitator and the candidate patient, a video conference between the healthcare facilitator and the candidate patient, an electronic chat session between the healthcare facilitator and the candidate patient, or an electronic message exchange between the healthcare facilitator and the candidate patient in any suitable combination. In still yet another embodiment of the process 100, the collecting of the first client information includes at least one of collecting personal identifying information (PII) from the community health client, collecting demographic information from the community health client, and collecting “reason for visit” information from the community health client. In another embodiment of the process 100, the selected clinical sequence includes a foot clinic workflow, a diabetes clinic workflow, a health and human performance clinic workflow, an infectious disease clinic workflow, or an asthma clinic workflow in any suitable combination.

With reference to FIGS. 1 and 2, another exemplary embodiment of a process 200 for improving healthcare through community engagement includes the process 100 of FIG. 1 and continues from 108 to 202 where the first client information from the engagement session with the community health client is at least temporarily stored in a storage device at the computing device. At 204, the first client information from the engagement session with the community health client is transmitted from the storage device at the computing device to a centralized community health information database of a server computing system. The server computing system being accessible to the computing device. At 206, the first client information from the engagement session with the community health client is stored in the centralized community health information database of the server computing system with other client information from other engagement sessions with the community health client. In another embodiment, the server computing system is configured to store first client information for other community health clients.

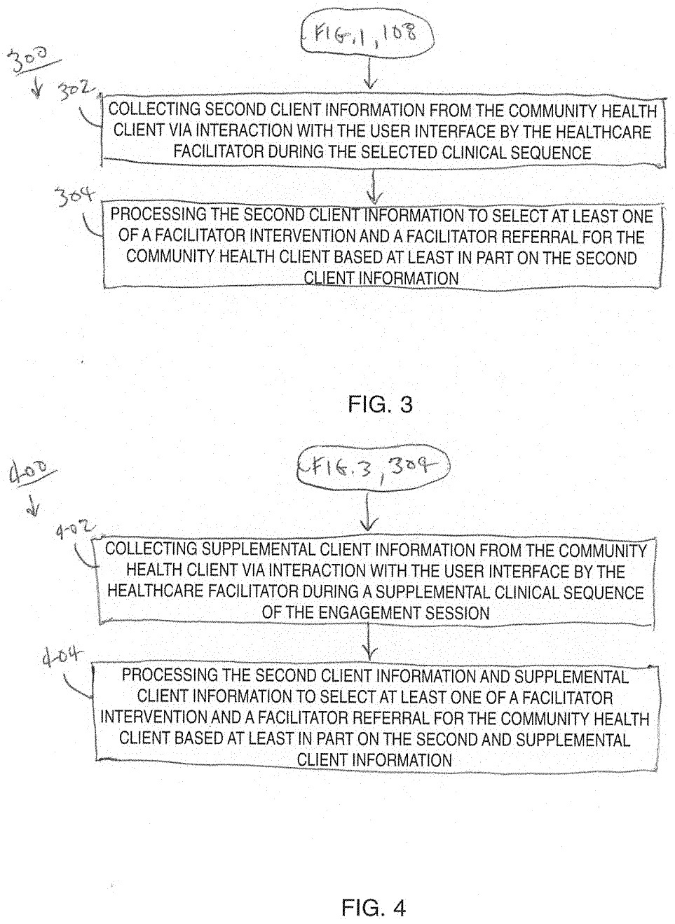

With reference to FIGS. 1 and 3, yet another exemplary embodiment of a process 300 for improving healthcare through community engagement includes the process 100 of FIG. 1 and continues from 108 to 302 where second client information is collected from the community health client via interaction with the user interface by the healthcare facilitator during the selected clinical sequence. The community health application controlling the user interface by presenting displays on the display device based on the selected clinical sequence and reading data from the user interface in relation to the displays. At 304, the second client information is processed to select at least one of a facilitator intervention and a facilitator referral for the community health client based at least in part on the second client information. As for selection of facilitator interventions, the community health application can select customized recommendations for education and other interventions based on gender, race, genetic risk factors, and other information collected in the first client information during the client identification sequence of the engagement session as well as health status and other information collected in the second client information during the clinical sequence. Thus, the community health application enables the engagement session to serve as a CDSS that can recommend education and other interventions for the community health client based on best practice guidelines.

In another embodiment of the process 300, the processing of the second client information includes determining if supplemental client information is needed for the engagement session with the community health client based at least in part on the second client information.

In yet another embodiment of the process 300, the collecting of the second client information includes collecting physiological information from the community health client, collecting a facilitator observation of the community health client, collecting a photographic image of the community health client, or collecting a biological sample from the community health client in any suitable combination. The biological sample may be collected via a cheek swab or any suitable biological sampling technique. The biological sample is preserved until it can be processed by appropriate instruments needed to perform the desired test. The result from processing the biological sample may be captured electronically and stored with other client information obtained through the engagement session. The processing of the biological sample may be at the same location as the engagement session, at an off-site laboratory, or any suitable combination thereof.

In a further embodiment, the biological sample is for glucose testing, infectious disease testing, DNA testing, or any suitable type of in vitro testing in any suitable combination. In an even further embodiment, the infectious disease testing includes viral infection testing, COVID-19 viral testing, coronavirus testing, annual flu testing, community pneumonia testing, bacterial infection testing, antibody testing, fungus testing, parasite testing, or any suitable type of in vitro infectious disease testing in any suitable combination. In another even further embodiment, the DNA testing may be used for genetic analysis of the client; epigenetic, pharmocogenetic, proteomics, or various types of genetic research on individuals or genome; and/or epigenomic research on groups of clients. The DNA samples may be collected via a cheek swab or any suitable DNA sampling technique. The DNA samples are preserved until they can be processed by appropriate instruments needed to obtain the desired genetic, epigenetic, or genomic information. The results from processing the DNA samples are captured electronically and stored with other client information obtained through the engagement session. The processing of the DNA samples may be at the same location as the engagement session, at various off-site laboratories, or any suitable combination thereof.

With reference to FIGS. 1, 3, and 4, still another exemplary embodiment of a process 400 for improving healthcare through community engagement includes the processes 100, 300 of FIGS. 1 and 3 and continues from 304 to 402 where supplemental client information is collected from the community health client via interaction with the user interface by the healthcare facilitator during a supplemental clinical sequence of the engagement session. The community health application controlling the user interface by presenting displays on the display device based on the supplemental client information needed and reading data from the user interface in relation to the displays. At 404, the second client information and supplemental client information is processed to select at least one of a facilitator intervention and a facilitator referral for the community health client based at least in part on the second and supplemental client information. As for selection of facilitator interventions, the community health application can select customized recommendation for education and other interventions based on gender, race, genetic risk factors, and other information collected in the first client information during the client identification sequence of the engagement session as well as health status and other information collected in the second and supplemental client information during the clinical and supplemental clinical sequences. Thus, the community health application enables the engagement session to serve as a CDSS that can recommend education and other interventions for the community health client based on best practice guidelines.

In another embodiment of the process 400, the collecting of the supplemental client information includes collecting supplemental physiological information from the community health client, collecting a supplemental facilitator observation of the community health client, collecting a supplemental photographic image of the community health client, or collecting a biological sample from the community health client in any suitable combination. The biological sample collected during the collecting of the supplemental client information may be collected in the same manner and used for the same purpose as the biological sample described above in conjunction with the collecting of the second client information. In this regard, it is understood that a biological sample may be collected with the second client information, with the supplemental client information, or with both second client information and supplemental client information.

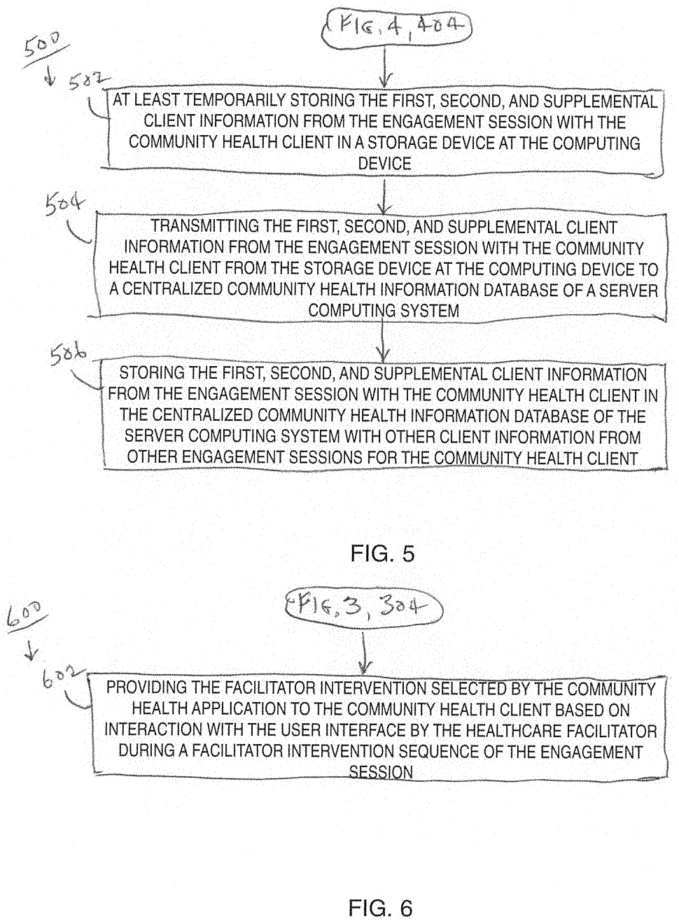

With reference to FIGS. 1, 3, and 5, still yet another exemplary embodiment of a process 500 for improving healthcare through community engagement includes the processes 100, 300 of FIGS. 1 and 3 and continues from 304 to 502 where the first, second, and supplemental client information from the engagement session with the community health client is at least temporarily stored in a storage device at the computing device. At 504, the first, second, and supplemental client information from the engagement session with the community health client is transmitted from the storage device at the computing device to a centralized community health information database of a server computing system. The server computing system being accessible to the computing device. At 506, the first, second, and supplemental client information from the engagement session with the community health client is stored in the centralized community health information database of the server computing system with other client information from other engagement sessions with the community health client. In another embodiment, the server computing system is configured to store first, second, and supplemental client information for other community health clients.

With reference to FIGS. 1, 3, and 6, another exemplary embodiment of a process 600 for improving healthcare through community engagement includes the processes 100, 300 of FIGS. 1 and 3 and continues from 304 to 602 where the facilitator intervention selected by the community health application is provided to the community health client based on interaction with the user interface by the healthcare facilitator during a facilitator intervention sequence of the engagement session. The community health application controlling the user interface by presenting displays on the display device based on the selected facilitator intervention and reading data from the user interface in relation to the displays. The healthcare facilitator performing the interventions identified in the displays and providing comments and feedback data regarding such interventions in corresponding fields of the displays. In another embodiment of the process 600, the providing of the facilitator intervention includes providing personal services to the community health client, providing personal goods to the community health client, or providing educational services to the community health client in any suitable combination. The facilitator intervention selected by the community health application can provide a customized recommendation for education and other interventions based on gender, race, genetic risk factors, and other information collected in the first client information during the client identification sequence of the engagement session as well as health status and other information collected in the second client information during the clinical sequence. Thus, the community health application enables the engagement session to serve as a CDSS that can recommend education and other interventions for the community health client based on best practice guidelines.

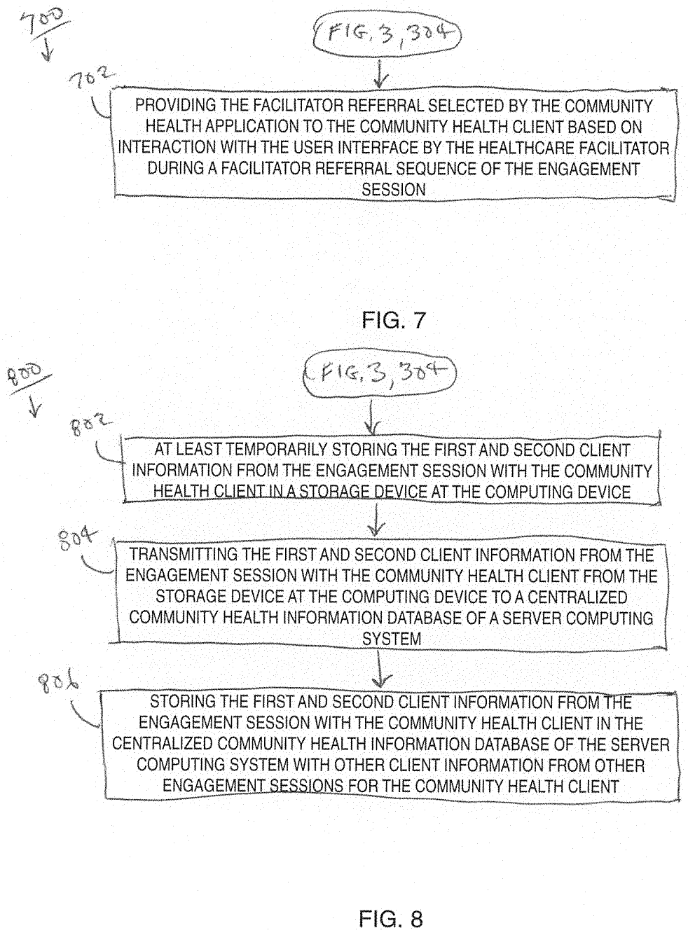

With reference to FIGS. 1, 3, and 7, another exemplary embodiment of a process 700 for improving healthcare through community engagement includes the processes 100, 300 of FIGS. 1 and 3 and continues from 304 to 702 where the facilitator referral selected by the community health application is provided to the community health client based on interaction with the user interface by the healthcare facilitator during a facilitator referral sequence of the engagement session. The community health application controlling the user interface by presenting displays on the display device based on the selected facilitator referral and reading data from the user interface in relation to the displays. The healthcare facilitator performing the referrals identified in the displays and providing comments and feedback data regarding such referrals in corresponding fields of the displays. In another embodiment of the process 700, the providing of the facilitator referral includes providing a report listing facilitator referrals to the community health client, providing a referral for healthcare services to the community health client, providing a referral for personal services to the community health client, providing a referral for personal goods to the community health client, or providing a referral for educational services to the community health client in any suitable combination.

In another embodiment of the process 700, the providing of the facilitator referral includes providing an on-site referral to the community health client. In this embodiment, the process 700 also includes sending an electronic message to an on-site healthcare provider to provide at least one of notice of the on-site referral to the on-site healthcare provider, at least a portion of the first and second client information from the engagement session with the community health client to the on-site healthcare provider, and at least a recommendation of a schedule for a referral session with the community health client to the on-site healthcare provider.

In a further embodiment, the process 700 also including at least temporarily storing patient information from the referral session with the community health client in a second storage device at a second computing device used by the on-site healthcare provider. The patient information from the referral session with the community health client is transmitted from the second storage device at the second computing device to a centralized community health information database of a server computing system, the server computing system being accessible to the second computing device. The patient information from the referral session with the community health client is stored in the centralized community health information database of the server computing system with the first and second client information for the engagement session with the community health client. In another embodiment, the server computing system is configured to store patient information for other community health clients.

In yet another embodiment of the process 700, the providing of the facilitator referral includes providing an off-site referral to the community health client. In this embodiment, the process 600 also includes sending an electronic message to an off-site healthcare provider to provide at least one of notice of the off-site referral to the off-site healthcare provider, at least a portion of the first and second client information from the engagement session with the community health client to the off-site healthcare provider, and at least a recommendation of a schedule for a referral session with the community health client to the off-site healthcare provider.

With reference to FIGS. 1, 3, and 8, yet another exemplary embodiment of a process 800 for improving healthcare through community engagement includes the processes 100, 300 of FIGS. 1 and 3 and continues from 304 to 802 where the first and second client information from the engagement session with the community health client is at least temporarily stored in a storage device at the computing device. At 804, the first and second client information from the engagement session with the community health client is transmitted from the storage device at the computing device to a centralized community health information database of a server computing system. The server computing system being accessible to the computing device. At 806, the first and second client information from the engagement session with the community health client is stored in the centralized community health information database of the server computing system with other client information from other engagement sessions with the community health client. In another embodiment, the server computing system is configured to store first and second client information for other community health clients.

Various exemplary embodiments of a non-transitory computer-readable medium are also provided. The non-transitory computer-readable medium stores program instructions that, when executed by at least one processor, cause a computerized system to perform various embodiments of a process (e.g., 100-800) for improving healthcare through community engagement as shown in FIGS. 1-8 and the corresponding paragraphs above describing the various embodiments.

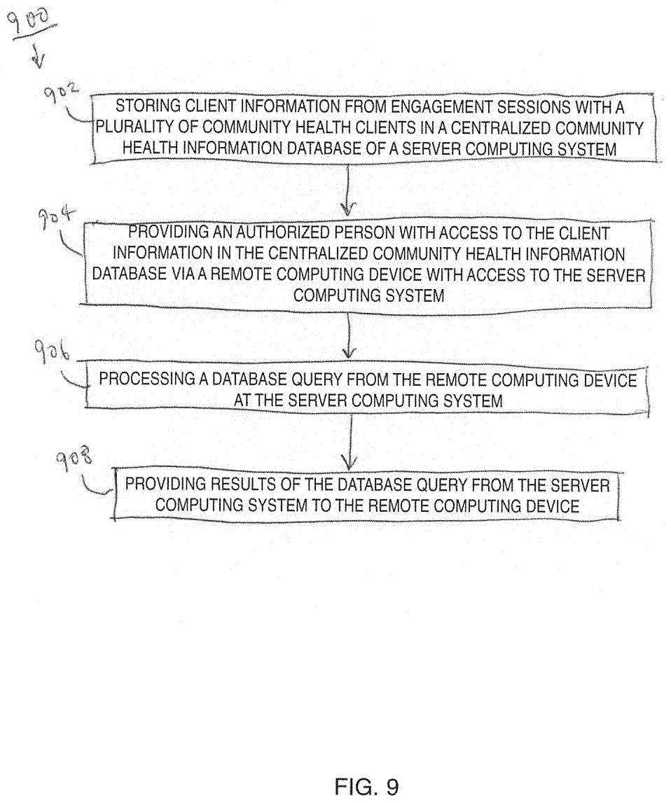

With reference to FIG. 9, an exemplary embodiment of a process 900 for improving healthcare through community engagement begins at 902 where client information from engagement sessions with a plurality of community health clients is stored in a centralized community health information database of a server computing system. The client information from the engagement sessions being captured electronically during the engagement sessions by healthcare facilitators using a community health application on computing devices. Each engagement session including a client identification sequence controlled by the community health application to identify the corresponding community health client, collect the client information from the community health client, and select a clinical sequence for the engagement session based on the client information. At 904, an authorized person is provided with access to the client information in the centralized community health information database via a remote computing device with access to the server computing system. For example, the authorized person may be provided with web-based access to the centralized community health information database. At 906, a database query from the remote computing device is processed at the server computing system. At 908, results of the database query are provided from the server computing system to the remote computing device. In another embodiment of the process 900, the providing of the results of the database query includes at least one of providing the authorized person with web-based results, providing the authorized person with the results in a printable electronic file, and providing the authorized person with the results in an electronic message.

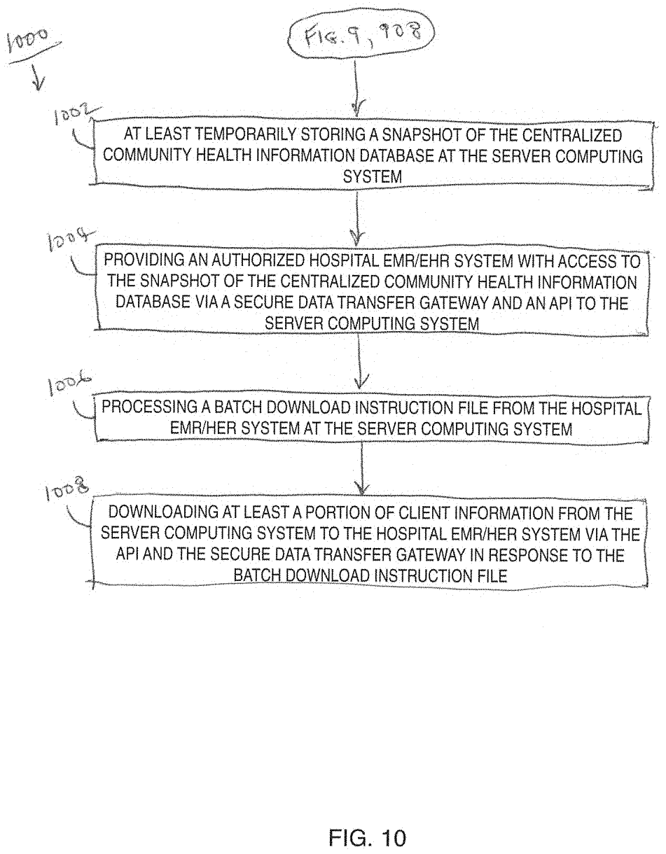

With reference to FIGS. 9 and 10, another exemplary embodiment of a process 1000 for improving healthcare through community engagement includes the process 900 of FIG. 9 and continues from 908 to 1002 where a snapshot of the centralized community health information database is at least temporarily stored at the server computing system. At 1004, an authorized hospital EMR/EHR system is provided with access to the snapshot of the centralized community health information database via a secure data transfer gateway and an application program interface (API) to the server computing system. At 1006, a batch download instruction file from the hospital EMR/EHR system is processed at the server computing system. At 1008, at least a portion of client information is downloaded from the server computing system to the hospital EMR/EHR system via the API and the secure data transfer gateway in response to the batch download instruction file.

With reference to FIGS. 9 and 11, yet another exemplary embodiment of a process 1100 for improving healthcare through community engagement includes the process 900 of FIG. 9 and continues from 908 to 1102 where a snapshot of at least a portion of the centralized community health information database is at least temporarily stored at the server computing system. At 1104, the client information in the snapshot is processed to de-identify community health clients associated with the client information to clean, transform, and enhance the client information to form anonymous community health information compatible with a data warehouse stored in a cloud-based computing system. At 1106, the anonymous community health information is uploaded from the server computing system to the data warehouse stored in the cloud-based computing system via a secure data transfer gateway.

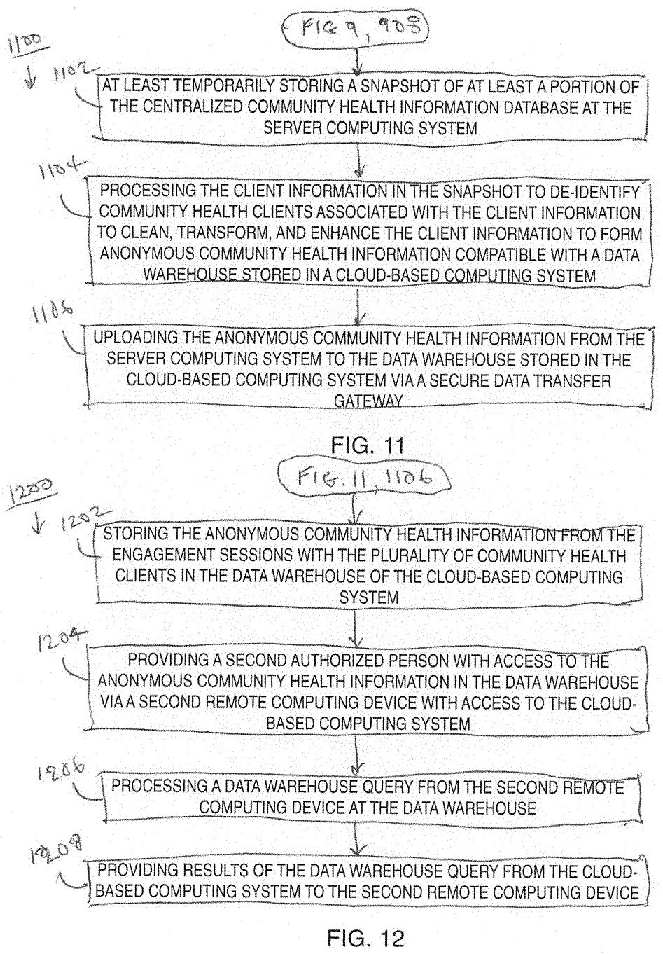

With reference to FIGS. 9, 11, and 12, still another exemplary embodiment of a process 1200 for improving healthcare through community engagement includes the processes 900, 1100 of FIGS. 9 and 11 and continues from 1106 to 1202 where anonymous community health information from engagement sessions with a plurality of community health clients is stored in the data warehouse of the cloud-based computing system. At 1204, a second authorized person is provided with access to the anonymous community health information in the data warehouse via a second remote computing device with access to the cloud-based computing system. For example, the second authorized person may be provided with web-based access to the data warehouse. At 1206, a data warehouse query from the second remote computing device is processed at the data warehouse. At 1208, results of the data warehouse query are provided from the cloud-based computing system to the second remote computing device. In another embodiment of the process 1200, the providing of the results of the data warehouse query include at least one of providing the second authorized person with web-based results, providing the second authorized person with the results in a printable electronic file, and providing the second authorized person with the results in an electronic message.

Various exemplary embodiments of another non-transitory computer-readable medium are also provided. This non-transitory computer-readable medium stores program instructions that, when executed by at least one processor, cause a computerized system to perform various embodiments of a process (e.g., 900-1200) for improving healthcare through community engagement as shown in FIGS. 9-12 and the corresponding paragraphs above describing the various embodiments.

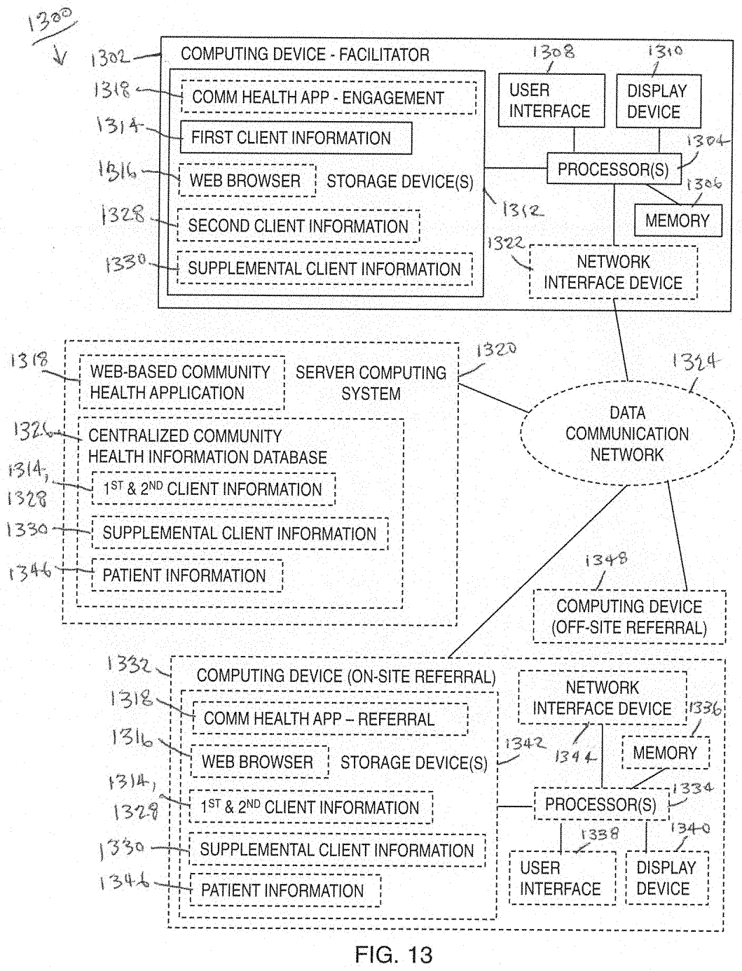

With reference to FIG. 13, an exemplary of embodiment of a computerized system 1300 for improving healthcare through community engagement includes a computing device 1302 configured for use by a healthcare facilitator for an engagement session with a candidate patient from a community. The computing device 1302 including at least one processor 1304 and associated memory 1306, a user interface 1308, a display device 1310, and at least one storage device 1312. The at least one processor 1304, associated memory 1306, user interface 1308, display device 1310, and at least one storage device 1312 are configured to initialize a community health application on the computing device 1302 for the engagement session. The at least one processor 1304, associated memory 1306, user interface 1308, display device 1310, and at least one storage device 1312 are configured to perform a client identification sequence using the community health application to enroll the candidate patient as a new community health client or lookup the candidate patient as an existing community health client. The at least one processor 1304, associated memory 1306, user interface 1308, display device 1310, and at least one storage device 1312 are configured to collect first client information 1314 from the community health client via interaction with the user interface 1308 of the computing device 1302 by the healthcare facilitator during the engagement session. The community health application controlling the user interface 1308 by presenting displays on the display device 1310 and reading data from the user interface 1308 in relation to the displays. The at least one processor 1304, associated memory 1306, and at least one storage device 1312 are configured to process the first client information 1314 to select a clinical sequence for the engagement session with the community health client based at least in part on the first client information 1314.

In another embodiment of the computerized system 1300, the community health application is a web-based application accessible to the computing device 1302 via a web browser 1316 stored on the at least one storage device 1312. In a further embodiment, the web-based application (i.e., community health application 1318) is stored on the at least one storage device 1312. In another further embodiment, the computerized system 1300 also includes a server computing system 1320 and the computing device 1302 also includes a network interface device 1322. The server computing system 1320 is configured to store the web-based application (i.e., community health application 1318). The web-based application (i.e., community health application 1318) is accessible to the computing device 1302 via the network interface device 1322. For example, the computing device 1302 and server computing system 1320 may be interconnected via a data communication network 1324 or any suitable communication path to enable access to the web-based application (i.e., community health application 1318).

In yet another embodiment of the computerized system 1300, the community health application 1318 is a computer-based application stored on the at least one storage device 1312.

In still another embodiment of the computerized system 1300, the at least one processor 1304, associated memory 1306, and at least one storage device 1312 are configured to at least temporarily store the first client information 1314 from the engagement session with the community health client in the at least one storage device 1312. In this embodiment, the computerized system 1300 also includes a server computing system 1320 configured to store a centralized community health information database 1326. The computing device 1302 also includes a network interface device 1322. The at least one processor 1304, associated memory 1306, at least one storage device 1312, and network interface device 1322 are configured to transmit the first client information 1314 from the engagement session with the community health client to the centralized community health information database 1326 of the server computing system 1320 via the network interface device 1322. For example, the computing device 1302 and server computing system 1320 may be interconnected via the data communication network 1324 or any suitable communication path for these transmissions. The server computing system 1320 is configured to store the first client information 1314 from the engagement session with the community health client in the centralized community health information database 1326 with other client information from other engagement sessions with the community health client. In another embodiment, the server computing system 1320 is configured to store first client information 1314 for other community health clients in the centralized community health information database 1326.

In still yet another embodiment of the computerized system 1300, the at least one processor 1304, associated memory 1306, user interface 1308, display device 1310, and at least one storage device 1312 are configured to collect second client information 1328 from the community health client via interaction with the user interface 1308 by the healthcare facilitator during the selected clinical sequence. The community health application 1318 controlling the user interface 1308 by presenting displays on the display device 1310 based on the selected clinical sequence and reading data from the user interface 1308 in relation to the displays. The at least one processor 1304, associated memory 1306, and at least one storage device 1312 are configured to process the second client information 1328 to select at least one of a facilitator intervention and a facilitator referral for the community health client based at least in part on the second client information 1328. As for selection of facilitator interventions, the community health application 1318 can select customized recommendation for education and other interventions based on gender, race, genetic risk factors, and other information collected in the first client information during the client identification sequence of the engagement session as well as health status and other information collected in the second client information during the clinical sequence. Thus, the community health application 1318 enables the engagement session to serve as a CDSS that can recommend education and other interventions for the community health client based on best practice guidelines.

In a further embodiment of the computerized system 1300, in conjunction with the processing of the second client information 1328, the at least one processor 1304, associated memory 1306, and at least one storage device 1312 are configured to determine if supplemental client information 1330 is needed for the engagement session with the community health client based at least in part on the second client information 1328.

In another further embodiment of the computerized system 1300, the at least one processor 1304, associated memory 1306, user interface 1308, display device 1310, and at least one storage device 1312 are configured to collect supplemental client information 1330 from the community health client via interaction with the user interface 1308 by the healthcare facilitator during a supplemental clinical sequence of the engagement session. The community health application 1318 controlling the user interface 1308 by presenting displays on the display device 1310 based on the supplemental client information 1330 needed and reading data from the user interface 1308 in relation to the displays. The at least one processor 1304, associated memory 1306, and at least one storage device 1312 are configured to process the second client information 1328 and supplemental client information 1330 to select at least one of a facilitator intervention and a facilitator referral for the community health client based at least in part on the second and supplemental client information. As for selection of facilitator interventions, the community health application can select customized recommendation for education and other interventions based on gender, race, genetic risk factors, and other information collected in the first client information during the client identification sequence of the engagement session as well as health status and other information collected in the second and supplemental client information during the clinical and supplemental clinical sequences. Thus, the community health application 1318 enables the engagement session to serve as a CDSS that can recommend education and other interventions for the community health client based on best practice guidelines.

In an even further embodiment of the computerized system 1300, the at least one processor 1304, associated memory 1306, and at least one storage device 1312 are configured to at least temporarily store the first, second, and supplemental client information 1314, 1328, 1330 from the engagement session with the community health client in the at least one storage device 1312. In this embodiment, the computerized system 1300 also includes a server computing system 1320 configured to store a centralized community health information database 1326 and the computing device 1302 also includes a network interface device 1322. The at least one processor 1304, associated memory 1306, at least one storage device 1312, and network interface device 1322 are configured to transmit the first, second, and supplemental client information 1314, 1328, 1330 from the engagement session with the community health client to the centralized community health information database 1326 of the server computing system 1320 via the network interface device 1322. For example, the computing device 1302 and server computing system 1320 may be interconnected via the data communication network 1324 or any suitable communication path for these transmissions. The server computing system 1320 is configured to store the first, second, and supplemental client information 1314, 1328, 1330 from the engagement session with the community health client in the centralized community health information database 1326 with other client information from other engagement sessions with the community health client. In another embodiment, the server computing system 1320 is configured to store first, second, and supplemental client information 1314, 1328, 1330 for other community health clients in the centralized community health information database 1326.

In yet another further embodiment of the computerized system 1300, the at least one processor 1304, associated memory 1306, user interface 1308, display device 1310, and at least one storage device 1312 are configured to provide at least a portion of the facilitator intervention selected by the community health application 1318 to the community health client based on interaction with the user interface 1308 by the healthcare facilitator during a facilitator intervention sequence of the engagement session. The community health application 1318 controlling the user interface 1308 by presenting displays on the display device 1310 based on the selected facilitator intervention and reading data from the user interface 1308 in relation to the displays. The healthcare facilitator performing the interventions identified in the displays and providing comments and feedback data regarding such interventions in corresponding fields of the displays. The facilitator intervention selected by the community health application 1318 can provide a customized recommendation for education and other interventions based on gender, race, genetic risk factors, and other information collected in the first client information during the client identification sequence of the engagement session as well as health status and other information collected in the second client information during the clinical sequence. Thus, the community health application 1318 enables the engagement session to serve as a CDSS that can recommend education and other interventions for the community health client based on best practice guidelines.

In still another further embodiment of the computerized system 1300, the at least one processor 1304, associated memory 1306, user interface 1308, display device 1310, and at least one storage device 1312 are configured to provide at least a portion of the facilitator referral selected by the community health application 1318 to the community health client based on interaction with the user interface 1308 by the healthcare facilitator during a facilitator referral sequence of the engagement session. The community health application 1318 controlling the user interface 1308 by presenting displays on the display device 1310 based on the selected facilitator referral and reading data from the user interface 1308 in relation to the displays. The healthcare facilitator performing the referrals identified in the displays and providing comments and feedback data regarding such referrals in, corresponding fields of the displays.

In an even further embodiment of the computerized system 1300, wherein, in conjunction with the providing of the at least a portion of the facilitator referral, the at least one processor 1304, associated memory 1306, user interface 1308, display device 1310, and at least one storage device 1312 are configured to provide an on-site referral to the community health client. In this embodiment, the computing device 1302 also includes a network interface device 1322. The at least one processor 1304, associated memory 1306, at least one storage device 1312, and network interface device 1322 are configured to send an electronic message to the on-site healthcare provider to provide at least one of notice of the on-site referral to the on-site healthcare provider, at least a portion of the first and second client information 1314, 1328 from the engagement session with the community health client to the on-site healthcare provider, and at least a recommendation of a schedule for a referral session with the community health client to the on-site healthcare provider. For example, the on-site healthcare provider may be able to access the electronic message from a second computing device 1332. The computing device 1302 and the second computing device 1332 having access to an electronic messaging service via a data communication network 1324 or any suitable communication path to enable the sending and receiving of the electronic message.

In an even yet further embodiment, the computerized system 1300 also includes a server computing system 1320 configured to store a centralized community health information database 1326 and a second computing device 1332 configured for use by the on-site healthcare provider for a referral session with the community health client. The second computing device 1332 including at least one second processor 1334 and associated second memory 1336, a second user interface 1338, a second display device 1340, at least one second storage device 1342, and a second network interface device 1344. The at least one second processor 1334, associated second memory 1336, and at least one second storage device 1342 are configured to at least temporarily store patient information 1346 from the referral session with the community health client in the at least one second storage device 1342. The at least one second processor 1334, associated second memory 1336, at least one second storage device 1342, and second network interface device 1344 are configured to transmit the patient information 1346 from the referral session with the community health client to the centralized community health information database 1326 of the server computing system 1320 via the second network interface device 1320. For example, the second computing device 1332 and server computing system 1320 may be interconnected via the data communication network 1324 or any suitable communication path for these transmissions. The server computing system 1320 is configured to store the patient information 1346 from the referral session with the community health client in the centralized community health information database 1326 with the first and second client information 1314, 1328 for the engagement session with the community health client. In another embodiment, the server computing system 1320 is configured to store patient information 1346 for other community health clients.

In another even yet further embodiment of the computerized system 1300, in conjunction with the providing of the at least a portion of the facilitator referral, the at least one processor 1304, associated memory 1306, user interface 1308, display device 1310, and at least one storage device 1312 are configured to provide an off-site referral to the community health client. In this embodiment, the computing device 1302 also includes a network interface device 1322. The at least one processor 1302, associated memory 1304, at least one storage device 1312, and network interface device 1322 are configured to send an electronic message to the off-site healthcare provider to provide at least one of notice of the off-site referral to the off-site healthcare provider, at least a portion of the first and second client information 1314, 1328 from the engagement session with the community health client to the off-site healthcare provider, and at least a recommendation of a schedule for a referral session with the community health client to the off-site healthcare provider. For example, the off-site healthcare provider may be able to access the electronic message from a third computing device 1348. The computing device 1302 and the third computing device 1348 having access to an electronic messaging service via a data communication network 1324 or any suitable communication path to enable the sending and receiving of the electronic message.

In still yet another embodiment of the computerized system 1300, the at least one processor 1304, associated memory 1306, and at least one storage device 1312 are configured to at least temporarily store the first and second client information 1314, 1328 from the engagement session with the community health client in the at least one storage device 1312. In this embodiment, the computerized system 1300 also includes a server computing system 1320 configured to store a centralized community health information database 1326 and the computing device 1302 also includes a network interface device 1322. The at least one processor 1304, associated memory 1306, at least one storage device 1312, and network interface device 1322 are configured to transmit the first and second client information 1314, 1328 from the engagement session with the community health client to the centralized community health information database 136 of the server computing system 1320 via the network interface device 1322. For example, the computing device 1302 and server computing system 1320 may be interconnected via the data communication network 1324 or any suitable communication path for these transmissions. The server computing system 1320 is configured to store the first and second client information 1314, 1328 from the engagement session with the community health client in the centralized community health information database 1326 with other client information from other engagement sessions with the community health client. In another embodiment, the server computing system 1320 is configured to store first and second client information 1314, 1328 for other community health clients in the centralized community health information database.

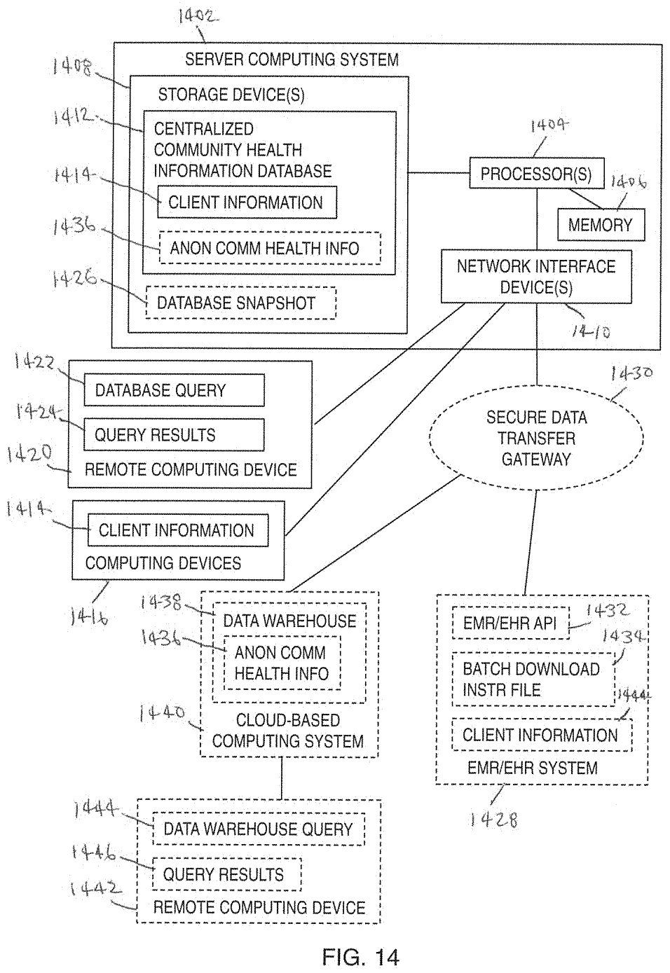

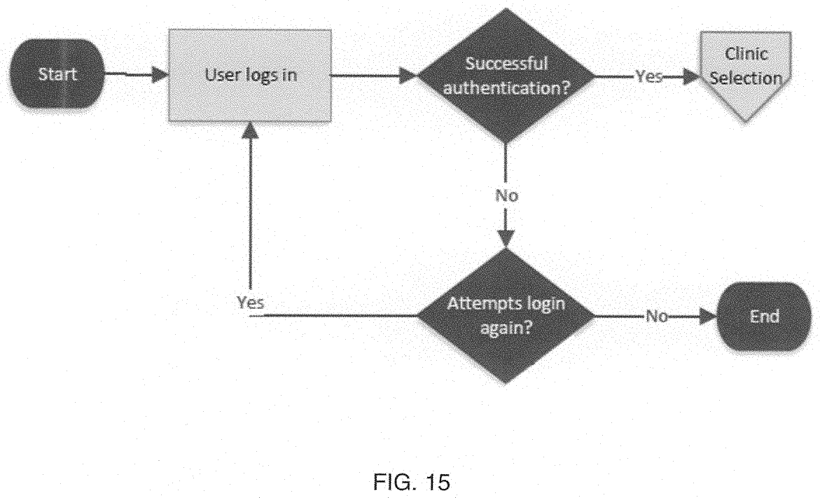

With reference to FIG. 14, an exemplary of embodiment of a computerized system 1400 for improving healthcare through community engagement includes a server computing device 1402 with at least one processor 1404 and associated memory 1406, at least one storage device 1408, and at least one network interface device 1410. The at least one storage device 1408 configured to store a centralized community health information database 1412. The at least one processor 1404 and associated memory 1406 and the at least one storage device are configured to store client information 1414 from engagement sessions with a plurality of community health clients in the centralized community health information database 1412. The client information 1414 from the engagement sessions being captured electronically during the engagement sessions by healthcare facilitators using a community health application on computing devices 1416. Each engagement session including a client identification sequence controlled by the community health application to identify the corresponding community health client, collect the client information 1414 from the community health client, and select a clinical sequence for the engagement session based on the client information 1414. The computing devices 1416 being configured to transmit the client information 1414 from the engagement sessions to the server computing system 1402 and the server computing system 1402 being configured to receive the client information 1414 via the at least one network interface device 1410. For example, the computing devices 1416 and server computing system 1402 may be interconnected via a data communication network or any suitable communication path for these transmissions. The at least one processor 1404 and associated memory 1406, the at least one storage device 1408, and the at least one network interface device 1410 are configured to provide an authorized person with access to the client information 1414 in the centralized community health information database 1412 via a remote computing device 1420. For example, the remote computing device 1420 and server computing system 1402 may be interconnected via a data communication network or any suitable communication path to enable access to the client information 1414. The at least one processor 1404 and associated memory 1406 and the at least one storage device 1408 are configured to process a database query 1422 from the remote computing device 1420 at the server computing system 1402. The at least one processor 1404 and associated memory 1406, the at least one storage device 1408, and the at least one network interface device 1410 are configured to provide results 1424 (i.e., query results) of the database query 1422 from the server computing system 1402 to the remote computing device 1420.

In another embodiment of the computerized system 1400, in conjunction with the providing of the results 1424 of the database query 1422, the at least one processor 1404 and associated memory 1406, the at least one storage device 1408, and the at least one network interface device 1410 are configured to perform at least one of providing the authorized person with web-based results, providing the authorized person with the results in a printable electronic file, and providing the authorized person with the results in an electronic message.

In yet another embodiment of the computerized system 1400, the at least one processor 1404 and associated memory 1406 and the at least one storage device 1408 are configured to at least temporarily store a snapshot 1424 (i.e., database snapshot) of the centralized community health information database 1412 at the server computing system 1402. The at least one processor 1404 and associated memory 1406, the at least one storage device 1408, and the at least one network interface device 1410 are configured to provide an authorized hospital EMR/EHR system 1428 with access to the snapshot 1426 of the centralized community health information database 1412 via a secure data transfer gateway 1430 and an API 1432 to the server computing system 1402. The at least one processor 1404 and associated memory 1406 and the at least one storage device 1408 are configured to process a batch download instruction file 1434 from the hospital EMR/EHR system 1428 at the server computing system 1402. The at least one processor 1404 and associated memory 1406, the at least one storage device 1408, and the at least one network interface device 1410 are configured to download at least a portion of client information 1414 from the server computing system 1402 to the hospital EMR/EHR system 1428 via the API 1432 and the secure data transfer gateway 1430 in response to the batch download instruction file 1434.

In still another embodiment of the computerized system 1400, the at least one processor 1404 and associated memory 1406 and the at least one storage device 1408 are configured to at least temporarily store a snapshot 1426 (i.e., database snapshot) of at least a portion of the centralized community health information database 1412 at the server computing system 1402. The at least one processor 1404 and associated memory 1406 and the at least one storage device 1408 are configured to process the client information 1414 in the snapshot 1426 to de-identify community health clients associated with the client information 1414 to clean, transform, and enhance the client information to form anonymous community health information 1436 compatible with a data warehouse 1438 stored in a cloud-based computing system 1440. The at least one processor 1404 and associated memory 1406, the at least one storage device 1408, and the at least one network interface device 1410 are configured to upload the anonymous community health information 1436 from the server computing system 1402 to the data warehouse 1438 stored in the cloud-based computing system 1440 via a secure data transfer gateway 1430.

In a further embodiment of the computerized system 1400, the data warehouse 1438 of the cloud-based computing system 1440 is configured to store the anonymous community health information 1436 from the engagement sessions with the plurality of community health clients. The data warehouse 1438 of the cloud-based computing system 1440 is configured to provide a second authorized person with access to the anonymous community health information 1436 in the data warehouse 1438 via a second remote computing device 1442 with access to the cloud-based computing system 1440. For example, the second remote computing device 1442 interconnected via a data communication network or any suitable communication path for access to the cloud-based computing system 1440. The data warehouse 1438 of the cloud-based computing system 1440 is configured to process a data warehouse query 1444 from the second remote computing device 1442. The data warehouse 1438 of the cloud-based computing system 1440 is configured to provide results 1446 (i.e., query results) of the data warehouse query 1444 to the second remote computing device 1442. In another embodiment, in conjunction with the providing of the results 1446 of the data warehouse query 1444, the data warehouse 1438 of the cloud-based computing system 1440 is configured to perform at least one of providing the second authorized person with web-based results, providing the second authorized person with the results in a printable electronic file, and providing the second authorized person with the results in an electronic message.

With reference to FIGS. 15 through 20, flow charts of exemplary processes for an exemplary client identification sequence in an engagement session with a candidate patient from a community are provided. FIG. 15 shows a process that enables a healthcare facilitator to login to a community health application on a computing device for an engagement session with a candidate patient from a community. Upon successful authentication of the login attempt, the community health application advances to a client identification sequence. The client identification sequence includes selecting a community healthcare engagement clinic at which the healthcare facilitator is engaging with candidate patients.

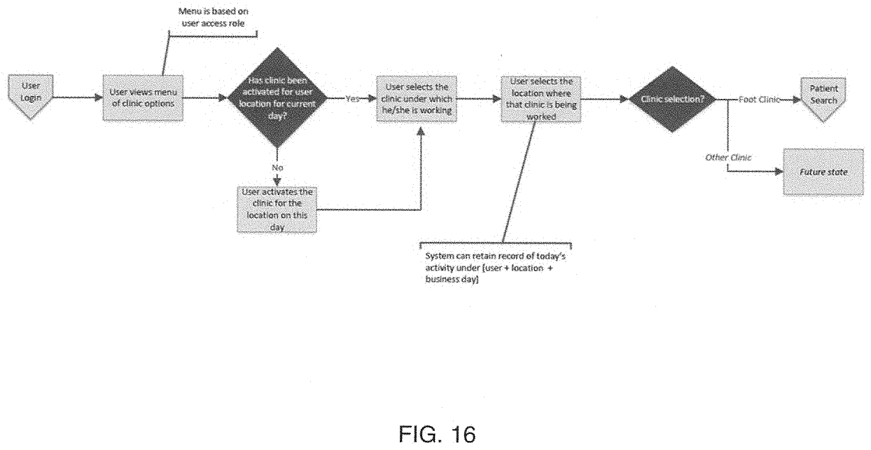

FIG. 16 shows a process that enables the healthcare facilitator to use the community health application to activate and/or select a specific community healthcare engagement clinic at which the healthcare facilitator is working. For example, the community healthcare engagement clinic may be located at a church, a library, a community center, a recreation center, or any other facility which has been made available for the clinic. The community healthcare engagement clinic could also be a virtual clinic that can be conducted using telemedicine techniques through a phone, video, and/or other virtual communication. After the specific community healthcare engagement clinic is identified, the community health application is ready for the healthcare facilitator to begin engaging with a candidate patient.

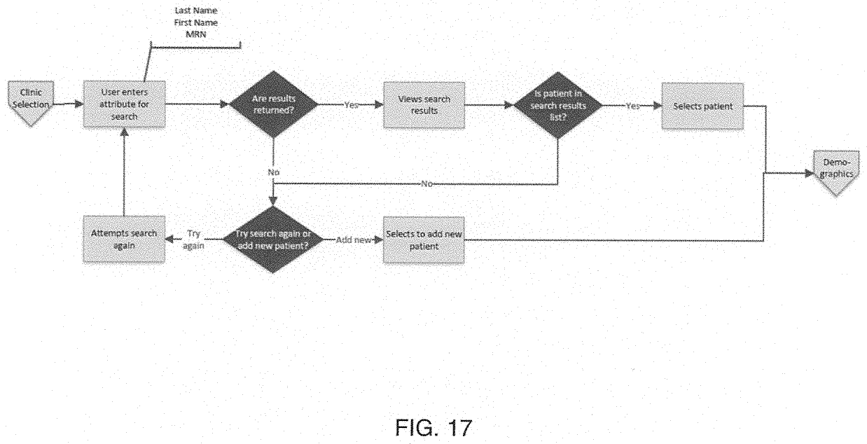

FIG. 17 shows a process that enables the healthcare facilitator to use the community health application to lookup a candidate patient if the patient is an existing community health client. The process also enables the healthcare facilitator to enroll the candidate patient as a new community health client.

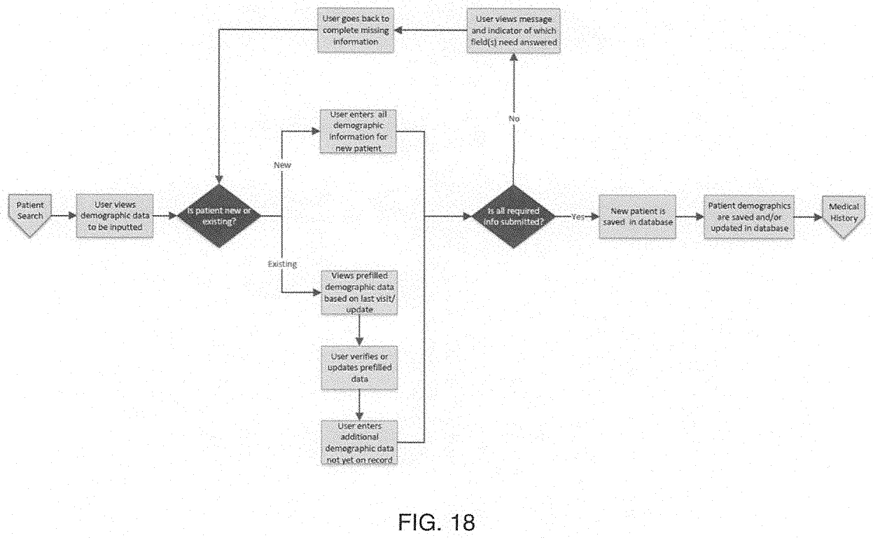

FIG. 18 shows a process that enables the healthcare facilitator to use the community health application to update demographic information for existing community health clients. The process also enables the healthcare facilitator to enter demographic information for new community health clients.

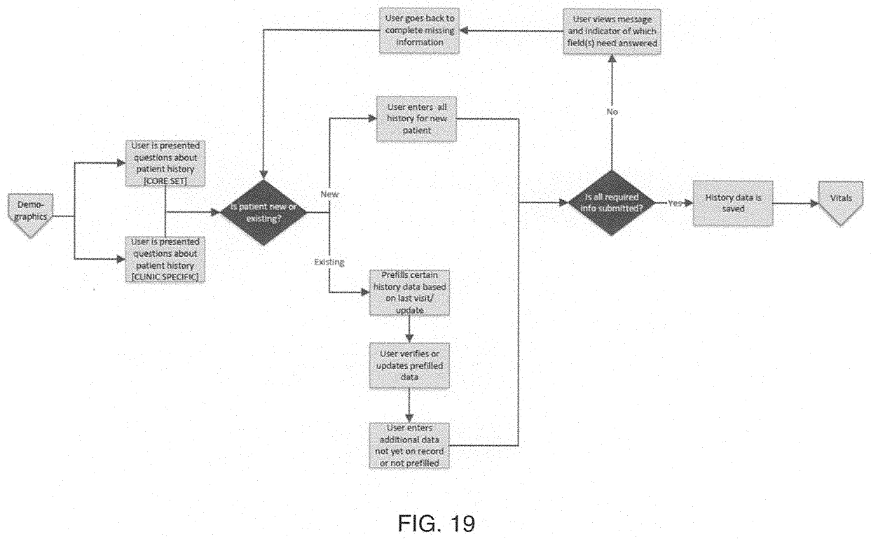

FIG. 19 shows a process that enables the healthcare facilitator to use the community health application to update medical history information for existing community health clients. The process also enables the healthcare facilitator to enter medical history information for new community health clients.

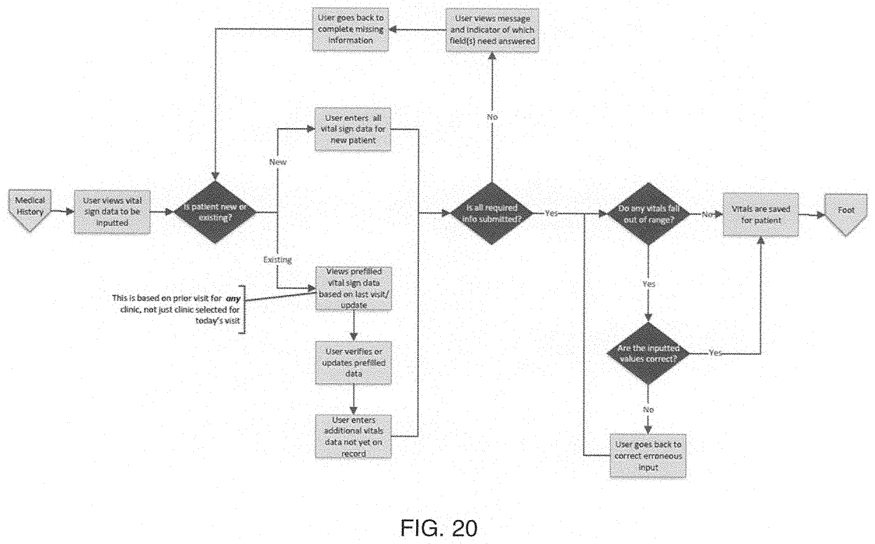

FIG. 20 shows a process that enables the healthcare facilitator to use the community health application to update vital sign information for existing community health clients. The process also enables the healthcare facilitator to enter vital sign information for new community health clients. If the specific community healthcare engagement clinic at which the community health client is enrolled offers more than one type of healthcare service, the process enables the healthcare facilitator to use the community health application to collect “reason for visit” information from the community health client. The community health application may select healthcare services for the community health client from those available at the engagement clinic based on the “reason for visit” and other information collected from the client. Alternatively, the healthcare facilitator may select the healthcare services for the community health client from those available at the engagement clinic.

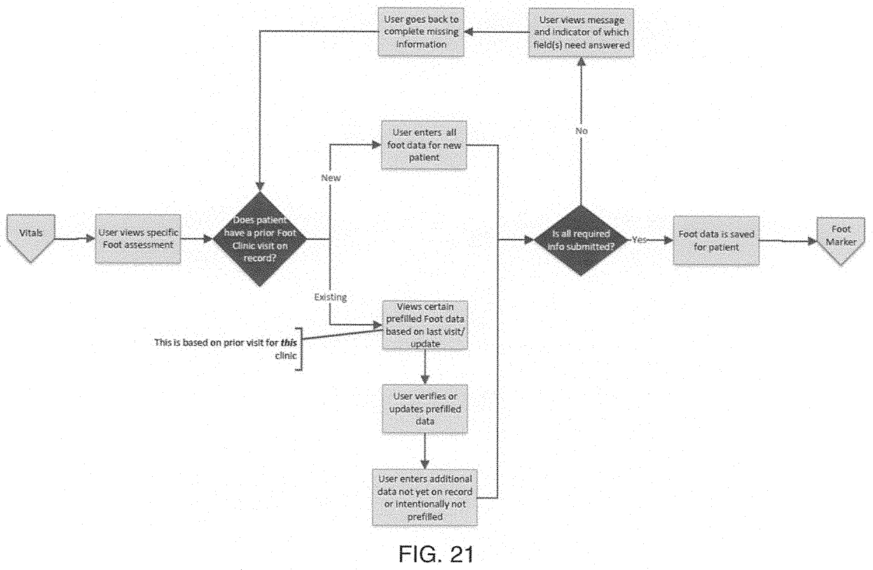

With reference to FIGS. 21 through 24, flow charts of exemplary processes for an exemplary clinical sequence in an engagement session with a community health client are provided. FIG. 21 shows a process that enables the healthcare facilitator to use the community health application to perform an assessment of the community health client for the clinical sequence. In this example, the clinical sequence is based on a workflow for a foot clinic. In other examples, the clinical sequence can be based on any suitable workflow for any suitable clinic or other type of healthcare service. The exemplary process enables the healthcare facilitator to use the community health application to perform a foot assessment of the community health client and to update foot data obtained during previous foot clinics for existing community health clients. The process also enables the healthcare facilitator to enter foot data for new community health clients.

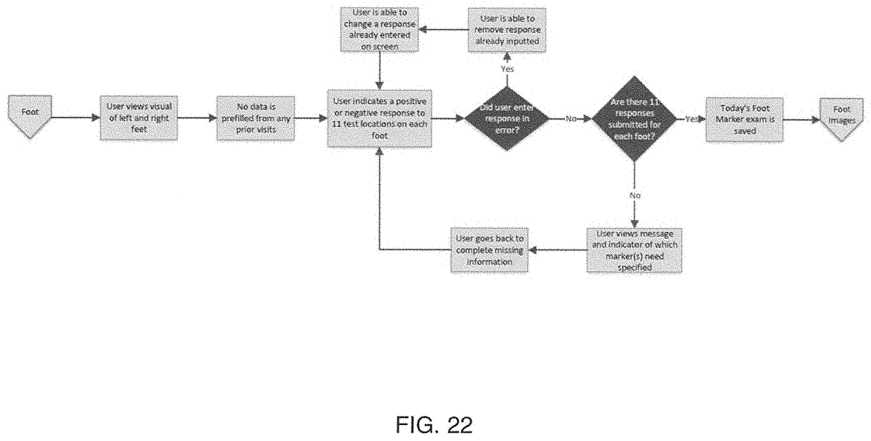

FIG. 22 shows a process that enables the healthcare facilitator to use the community health application to perform an examination of the community health client for the clinical sequence. In this example, the clinical sequence is based on a workflow for a foot clinic. In other examples, the clinical sequence can be based on any suitable workflow for any suitable clinic or other type of healthcare service. The exemplary process enables the healthcare facilitator to use the community health application to perform an examination of the community health client's feet to establish a marker for today's foot clinic.

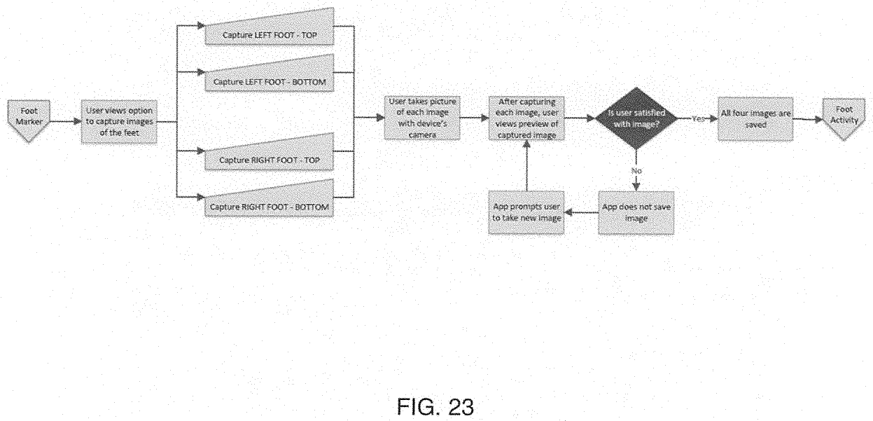

FIG. 23 shows a process that enables the healthcare facilitator to use the community health application to capture images of the community health client relating to the clinical sequence. In this example, the clinical sequence is based on a workflow for a foot clinic. In other examples, the clinical sequence can be based on any suitable workflow for any suitable clinic or other type of healthcare service. The exemplary process enables the healthcare facilitator to use the community health application to capture images of the tops and bottoms of the community health client's feet.

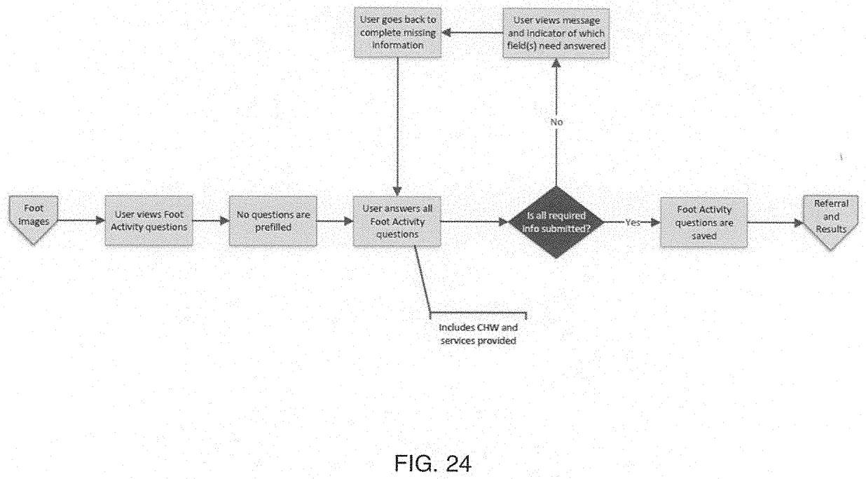

FIG. 24 shows a process that enables the healthcare facilitator to use the community health application to collect responses to predetermined activity questions relating to the clinical sequence from the community health client. In this example, the clinical sequence is based on a workflow for a foot clinic. In other examples, the clinical sequence can be based on any suitable workflow for any suitable clinic or other type of healthcare service. The exemplary process enables the healthcare facilitator to use the community health application to collect responses to predetermined foot activity questions from the community health client.

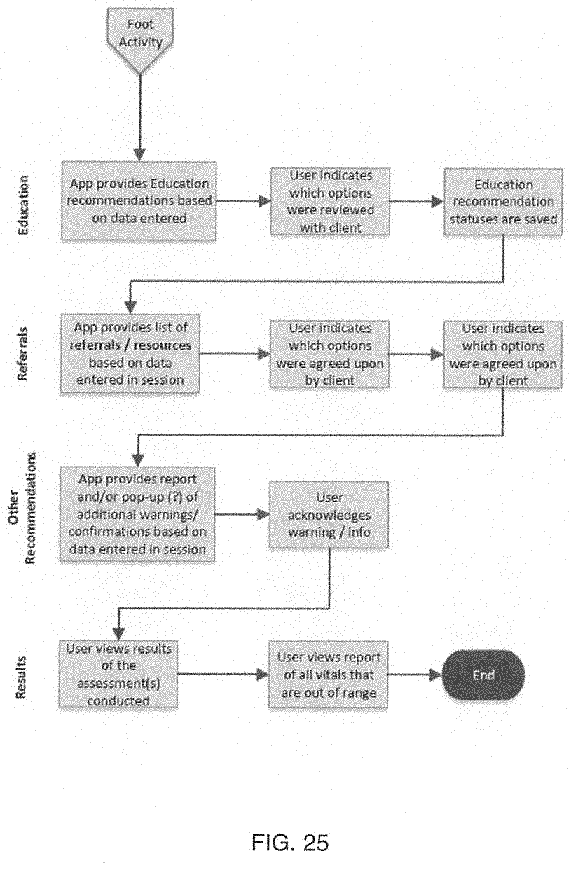

With reference to FIG. 25, a flow chart of exemplary facilitator intervention and facilitator referral sequences in an engagement session with a community health client is provided. The process enables the healthcare facilitator to use the community health application to provide interventions and referrals to the community health client based on the assessment, examination, and other information collected during the clinical sequence. In this example, the clinical sequence is based on a workflow for a foot clinic. In other examples, the clinical sequence can be based on any suitable workflow for any suitable clinic or other type of healthcare service. The exemplary process enables the healthcare facilitator to use the community health application to provide interventions to the community health client, such as education and other recommendations. The exemplary process also enables the healthcare facilitator to use the community health application to provide referrals to the community health client, such as referrals to on-site or off-site healthcare providers and referrals to other resources available to assist the client. The healthcare facilitator can use the community health application to capture the interventions and referrals provided to the community health client and to indicate those which were agreed to by the client. The healthcare facilitator can also use the community health application to provide a report summarizing the information captured during the clinical session to the community health client and to indicate that the report was provided to the client.

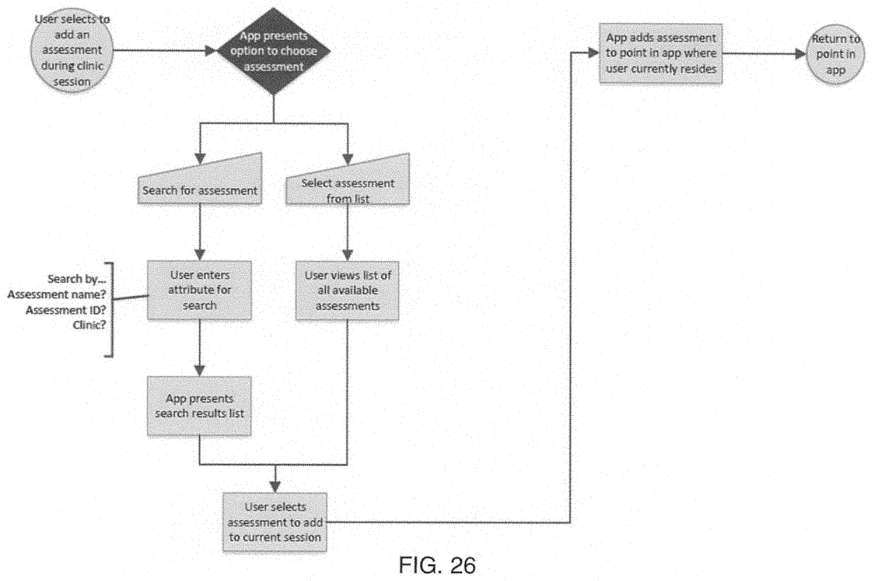

With reference to FIG. 26, a flow chart of an exemplary process for an exemplary supplemental clinical sequence in an engagement session with a community health client is provided. The process enables the healthcare facilitator to use the community health application to perform a supplemental assessment of the community health client for the clinical sequence. The community health application can activate the supplemental assessment process to capture additional information relating to the clinical sequence at any time during the clinical sequence. Similarly, the healthcare facilitator can activate the supplemental assessment process at any time during the clinical sequence.

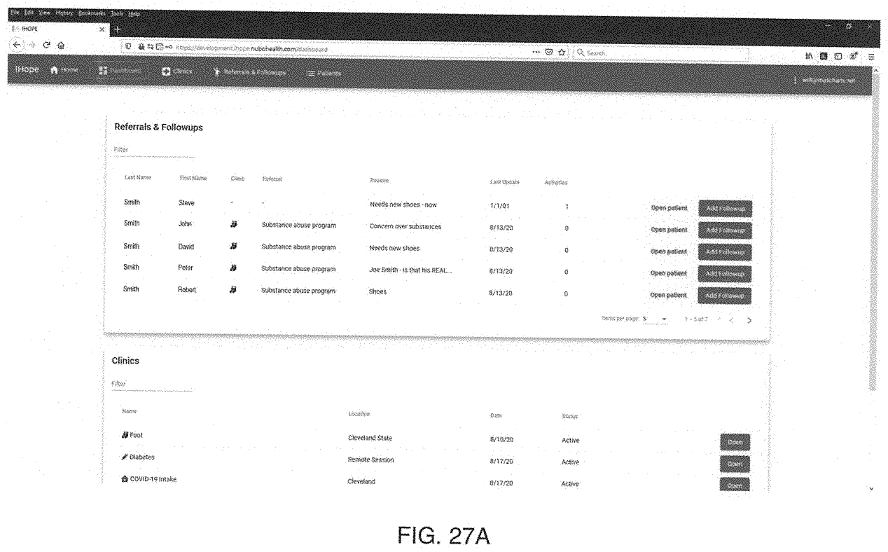

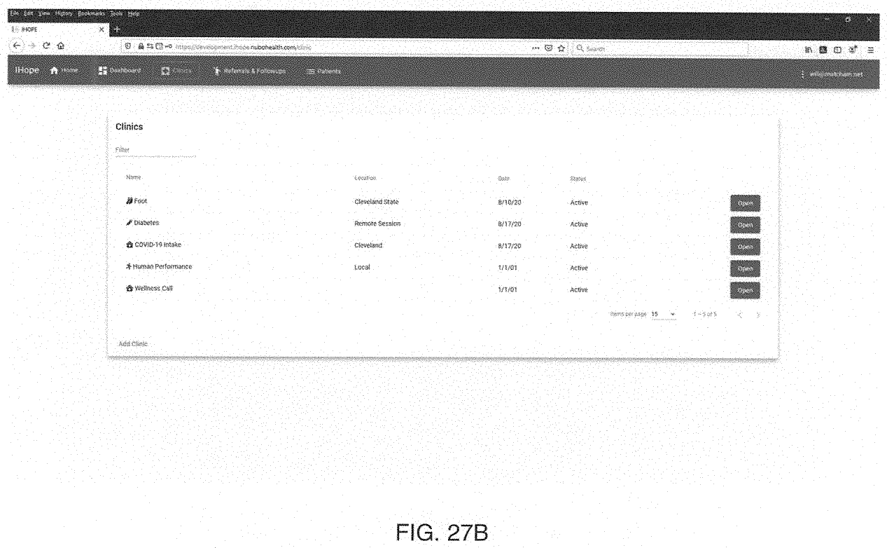

With reference to FIGS. 27A-B, exemplary dashboard and clinic displays presented on a computing device by an exemplary community health application are provided. FIG. 27A provides a dashboard display for an exemplary community health application. The dashboard display includes a menu bar with home, dashboard, clinics, referrals and followups, and patients menu items. In this exemplary display, the dashboard menu item is selected and the display shows a referral and followups panel and a clinics panel. In the exemplary display of FIG. 27B, the clinics menu item is selected and a list of clinics is provided in a clinics panel.

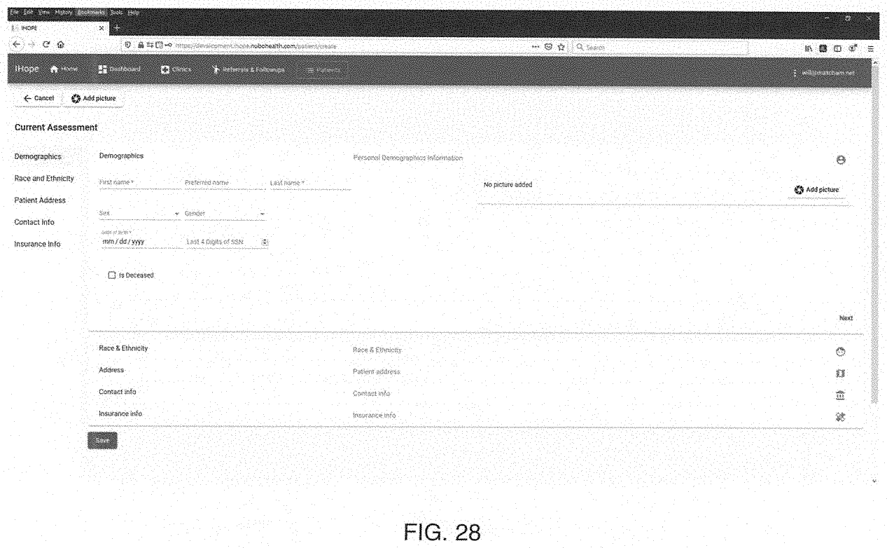

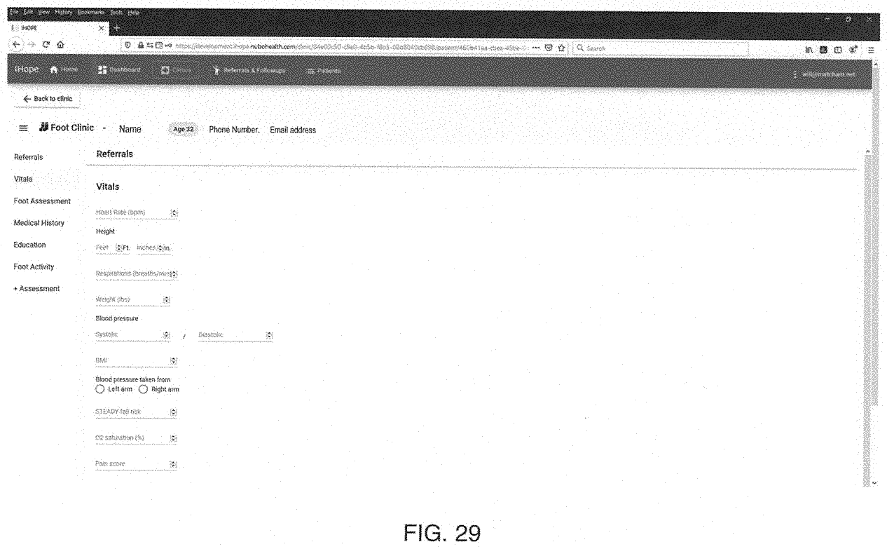

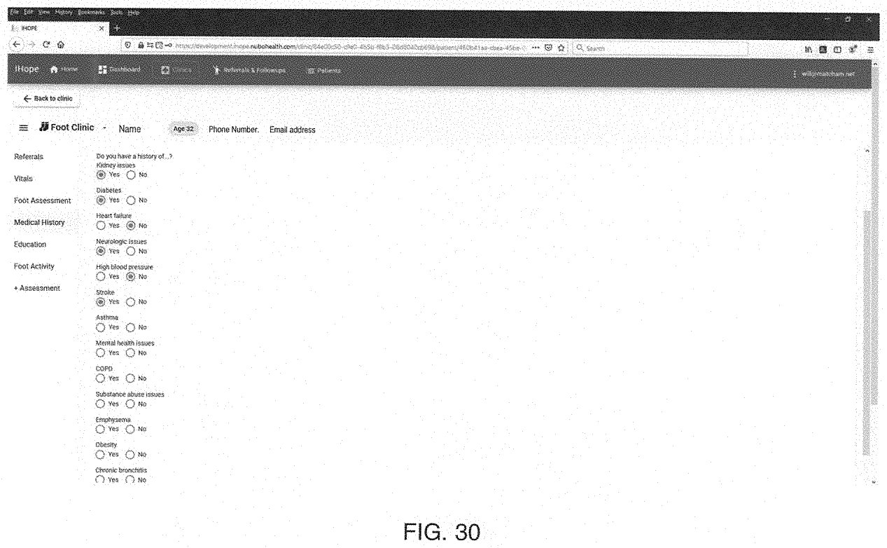

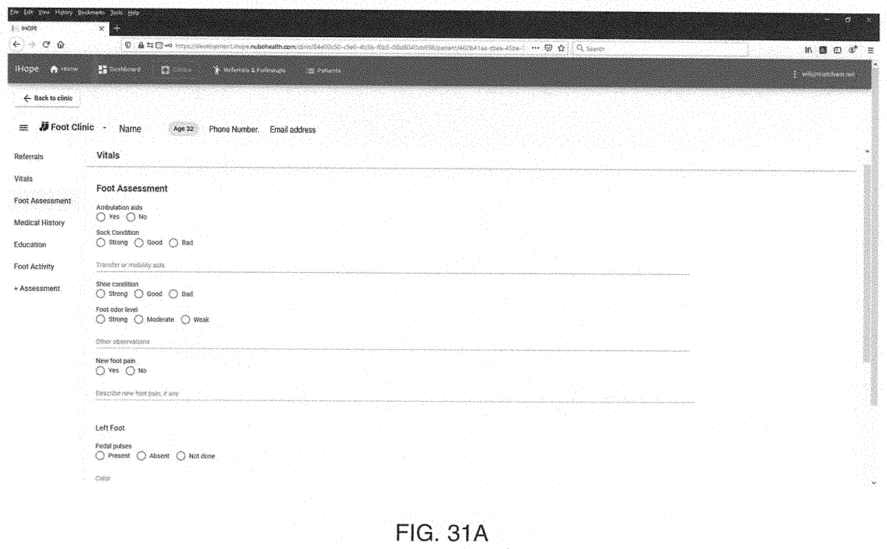

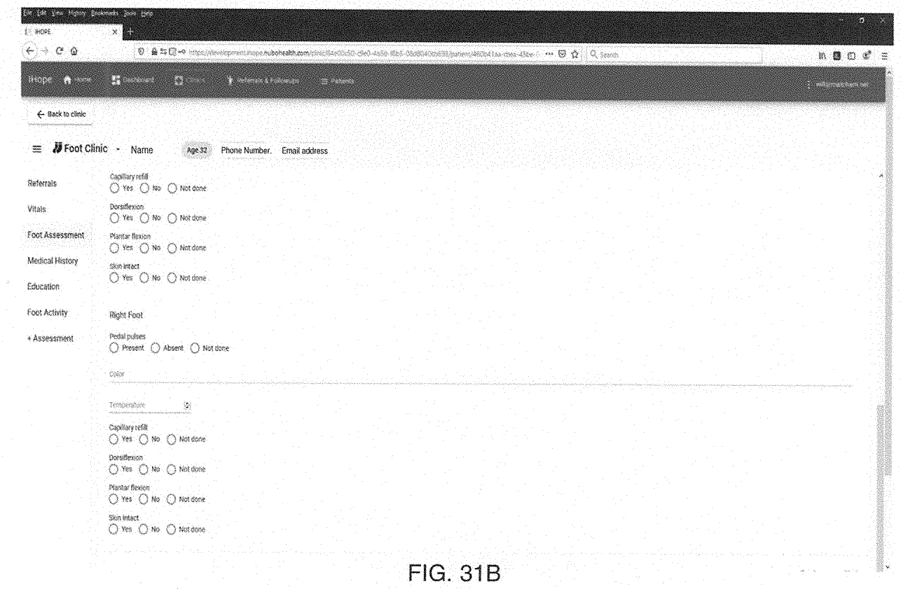

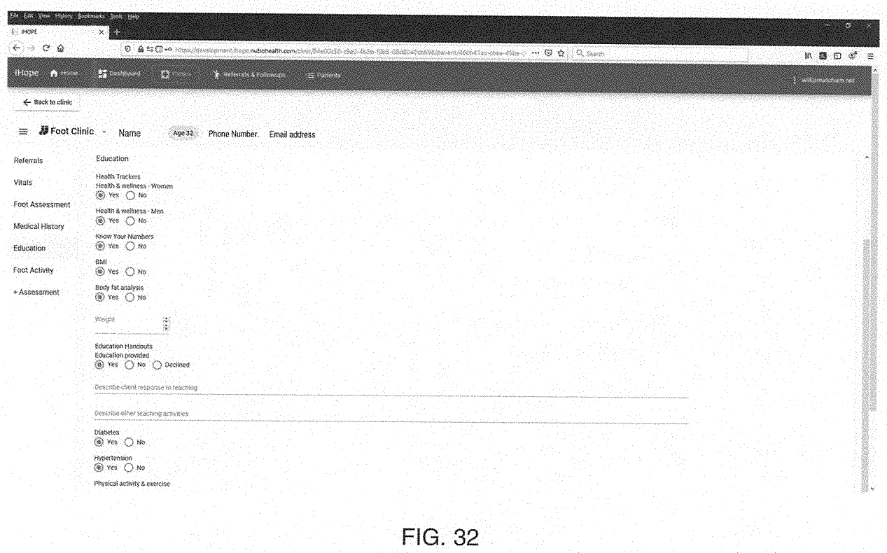

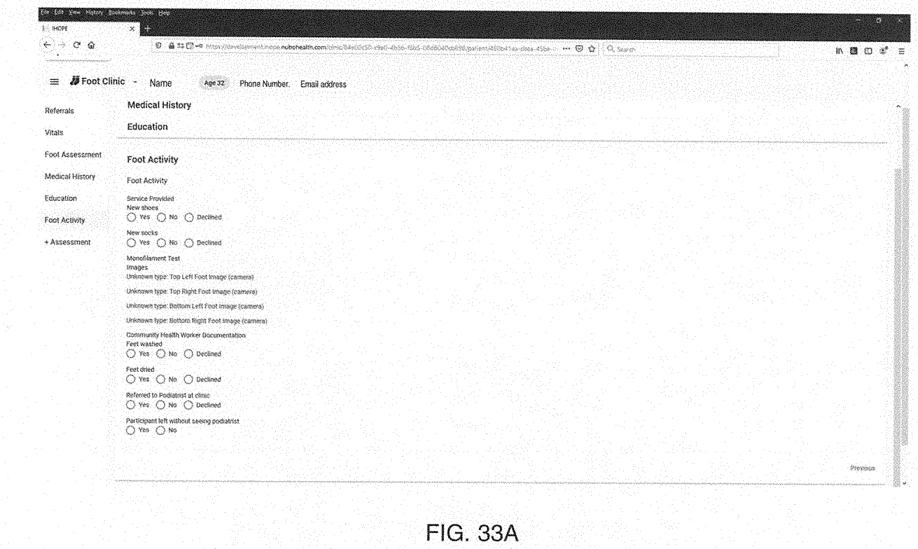

With reference to FIG. 28, an exemplary display for a demographics tab of a current assessment presented on a computing device by an exemplary community health application is provided.

With reference to FIG. 29, an exemplary display for a vitals tab of an exemplary foot clinic presented on a computing device by an exemplary community health application is provided.

With reference to FIG. 30, an exemplary display for a medical history tab of an exemplary foot clinic presented on a computing device by an exemplary community health application is provided.

With reference to FIGS. 31A-B, exemplary displays for a foot assessment tab of an exemplary foot clinic presented on a computing device by an exemplary community health application are provided. Notably, the display in FIG. 31B shows a display resulting from scrolling to the bottom of the inner scroll bar at the right side of the display shown in FIG. 31A which shows the scroll bar at the top.

With reference to FIG. 32, an exemplary display for an education tab of an exemplary foot clinic presented on a computing device by an exemplary community health application is provided.

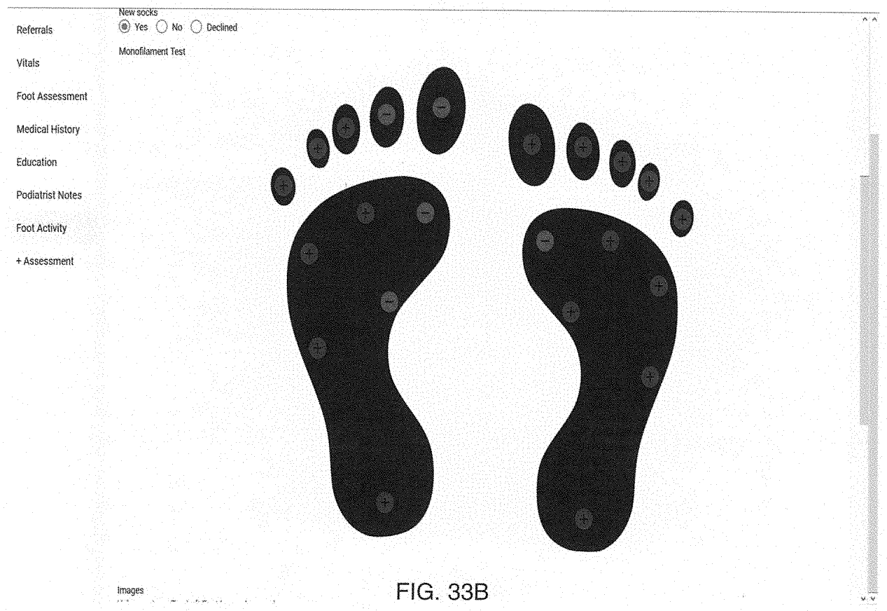

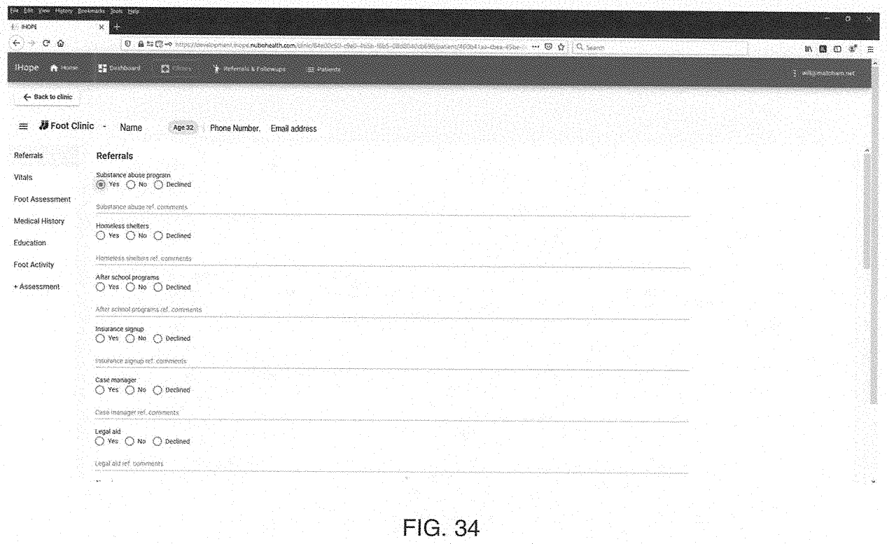

With reference to FIGS. 33A-B, exemplary displays for a foot activity tab of an exemplary foot clinic presented on a computing device by an exemplary community health application are provided. The display in FIG. 33A shows results of various foot activities the healthcare facilitator can provide to the community health client during the foot clinic. For example, the display in FIG. 33B shows the results of a monofilament test performed by the healthcare facilitator.

With reference to FIG. 34, an exemplary display for a referrals tab of an exemplary foot clinic presented on a computing device by an exemplary community health application is provided.

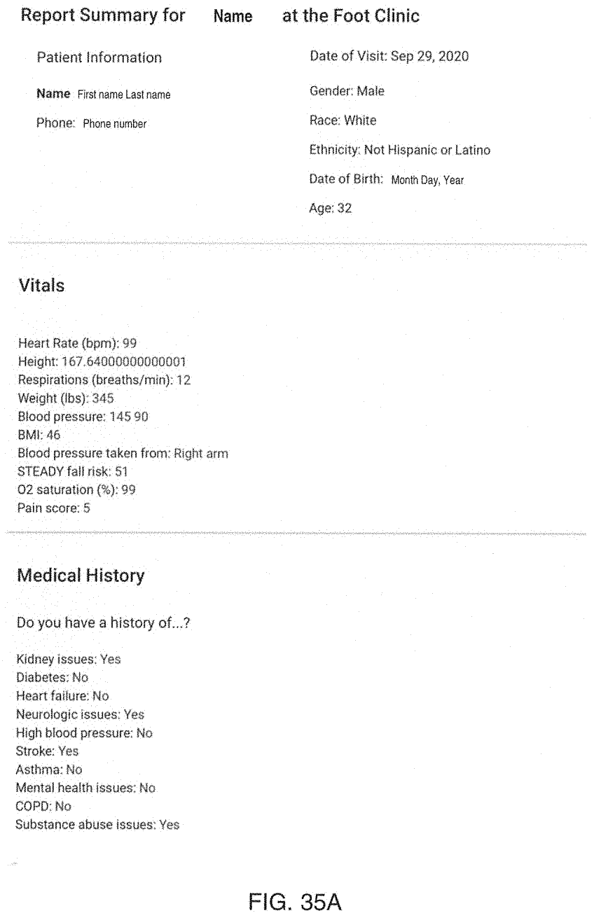

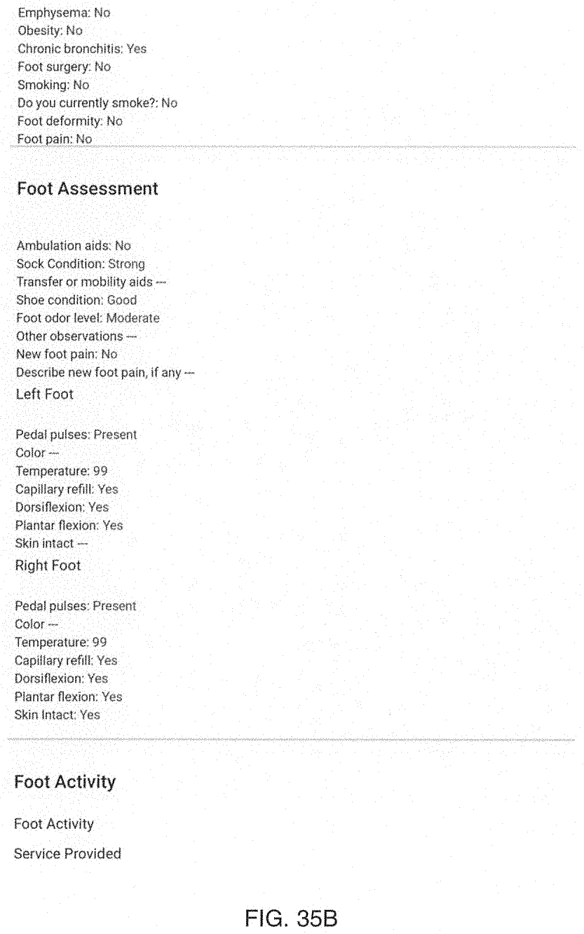

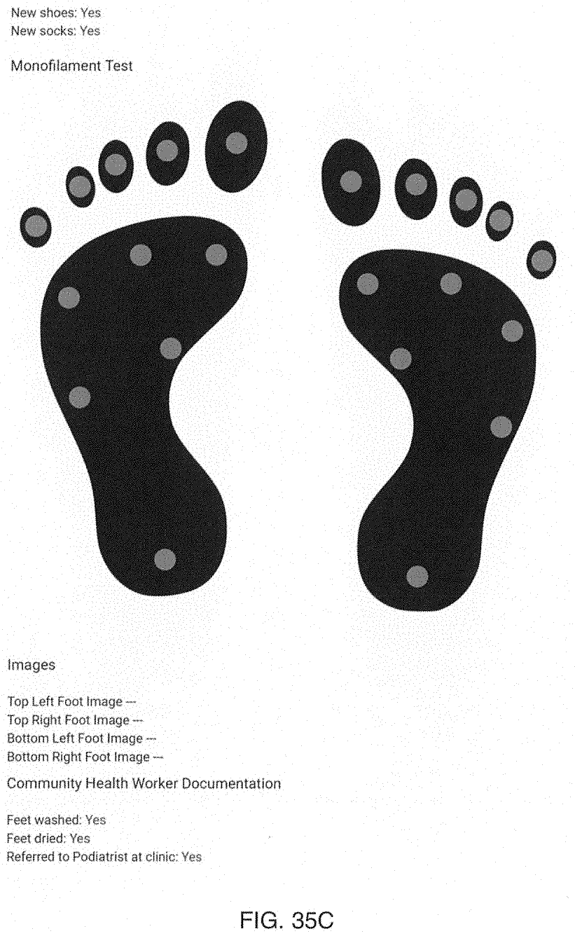

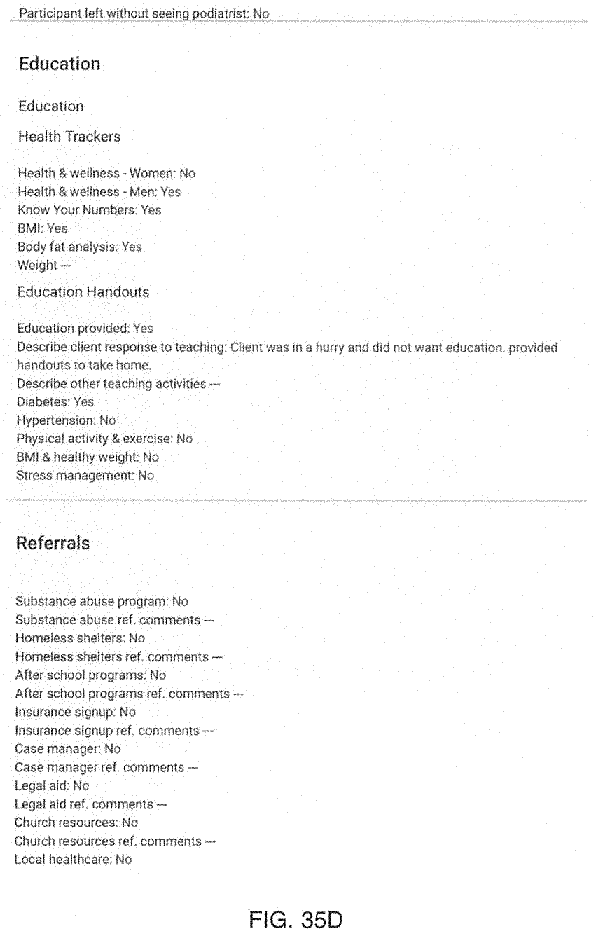

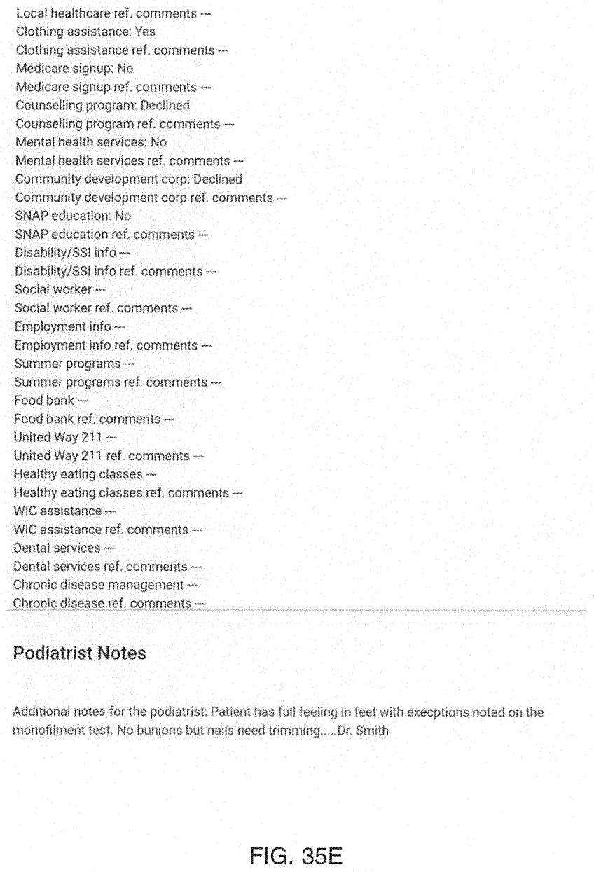

With reference to FIG. 35A-E, an exemplary report summary generated by an exemplary community health application on a computing device for an exemplary foot clinic is provided. The report summary includes identifying information for the community health client captured by the community health application during a client identification sequence. The report summary also includes information captured by the community health application during clinical, supplemental clinical, facilitator intervention, and facilitator referral sequences. In this example, the sequences are based on a workflow for a foot clinic. In other examples, the sequences can be based on any suitable workflow for any suitable clinic or other type of healthcare service. For example, the exemplary report summary for the foot clinic includes information sections for vitals, medical history, foot assessment, foot activity, education, referrals, and podiatrist notes.

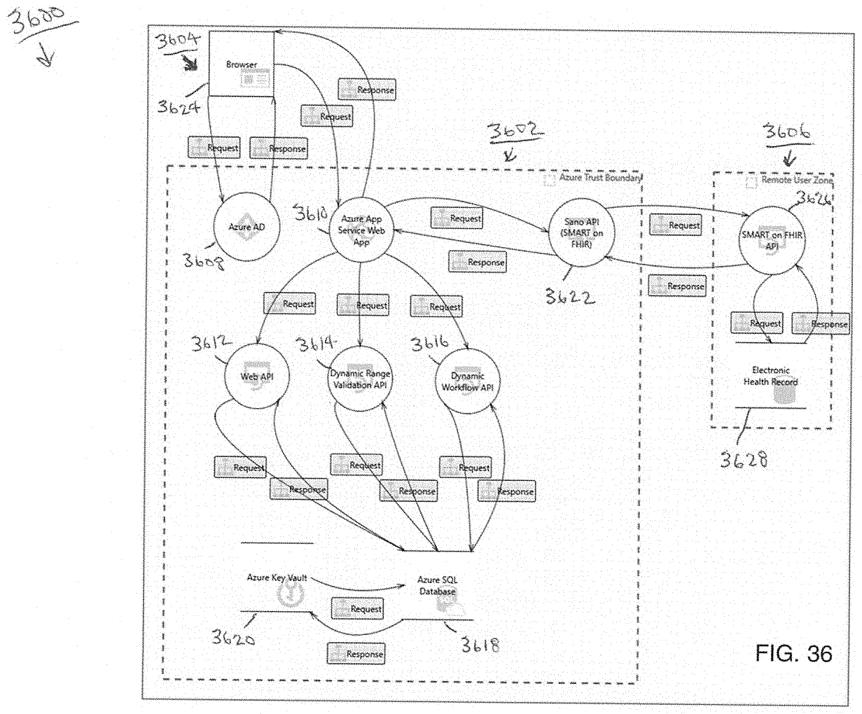

With reference to FIG. 36, yet another exemplary system 3600 for improving healthcare through community engagement includes a server computing system 3602, a computing device 3604, and an EMR/EHR system 3606. In this embodiment, the server computing system 3602 provides a cloud computing service using various features of Microsoft's Azure cloud computing service. The server computing system 3602 includes an Azure active directory 3608, an Azure app service web app 3610, a web API 3612, a dynamic range validation API 3614, a dynamic workflow API 3616, an Azure SQL database 3618, and an Azure key vault 3620. The server computing system 3602 also includes an EMR/EHR API 3622. In this embodiment, the EMR/EHR API 3622 includes a Sano API from nuboHealth that is compatible with a substitutable medical applications and reusable technologies (SMART) on fast health interoperability resources (FHIR) platform.

The computing device 3604 includes a browser 3624 configured to communicate with the Azure active directory 3608 and Azure app service web app 3610. The Azure active directory 3608 is configured to respond to requests from the browser 3624. The Azure app service web app 3610 is also configured to respond to requests from the browser 3624.

The EMR/EHR system 3606 includes a SMART on FHIR API 3626 and an EHR database 3628. The Azure app service web app 3610 is configured to communicate with the EHR database 3628 via the EMR/HER API 3622 and SMART on FHIR API 3626. The EHR database 3628 is configured to respond to requests from the Azure app service web app 3610 via the SMART on FHIR API 3626 and EMR/HER API 3622.

It will be appreciated that variants of the above-disclosed and other features and functions, or alternatives thereof, may be combined into many other different computer platforms, computer applications, or combinations thereof. Various presently unforeseen or unanticipated alternatives, modifications, variations, or improvements therein may be subsequently made by those skilled in the art which are also intended to be encompassed by the following claims.

What is claimed is:

1. A method for improving healthcare through community engagement, comprising:
initializing a community health application on a computing device for an engagement session with a candidate patient from a community;
performing a client identification sequence using the community health application to enroll the candidate patient as a new community health client or lookup the candidate patient as an existing community health client;
collecting first client information from the community health client via interaction with a user interface of the computing device by a healthcare facilitator during the engagement session, the community health application controlling the user interface by presenting displays on a display device and reading data from the user interface in relation to the displays; and
processing the first client information to select a clinical sequence for the engagement session with the community health client based at least in part on the first client information.

2. The method of claim 1, wherein the selected clinical sequence includes at least one of a foot clinic workflow, a diabetes clinic workflow, a health and human performance clinic workflow, an infectious disease clinic workflow, and an asthma clinic workflow.

3. The method of claim 1, further comprising:
at least temporarily storing the first client information from the engagement session with the community health client in a storage device at the computing device;
transmitting the first client information from the engagement session with the community health client from the storage device at the computing device to a centralized community health information database of a server computing system, the server computing system being accessible to the computing device; and
storing the first client information from the engagement session with the community health client in the centralized community health information database of the server computing system with other client information from other engagement sessions with the community health client.

4. The method of claim 1, further comprising:
collecting second client information from the community health client via interaction with the user interface by the healthcare facilitator during the selected clinical sequence, the community health application controlling the user interface by presenting displays on the display device based on the selected clinical sequence and reading data from the user interface in relation to the displays; and
processing the second client information to select at least one of a facilitator intervention and a facilitator referral for the community health client based at least in part on the second client information.

5. The method of claim 4, wherein the collecting of the second client information includes at least one of collecting physiological information from the community health client, collecting a facilitator observation of the community health client, collecting a photographic image of the community health client, and collecting a biological sample from the community health client;
wherein the biological sample is for at least one of glucose testing, infectious disease testing, and DNA testing;
wherein the infectious disease testing includes at least one of viral infection testing, COVID-19 viral testing, coronavirus testing, annual flu testing, community pneumonia testing, bacterial infection testing, antibody testing, fungus testing, and parasite testing.

6. The method of claim 4, further comprising:
collecting supplemental client information from the community health client via interaction with the user interface by the healthcare facilitator during a supplemental clinical sequence of the engagement session, the community health application controlling the user interface by presenting displays on the display device based on the supplemental client information needed and reading data from the user interface in relation to the displays; and
processing the second client information and supplemental client information to select at least one of a facilitator intervention and a facilitator referral for the community health client based at least in part on the second and supplemental client information.

7. The method of claim 6, further comprising:
at least temporarily storing the first, second, and supplemental client information from the engagement session with the community health client in a storage device at the computing device;
transmitting the first, second, and supplemental client information from the engagement session with the community health client from the storage device at the computing device to a centralized community health information database of a server computing system, the server computing system being accessible to the computing device; and
storing the first, second, and supplemental client information from the engagement session with the community health client in the centralized community health information database of the server computing system with other client information from other engagement sessions with the community health client.

8. The method of claim 4, further comprising:
providing the facilitator intervention selected by the community health application to the community health client based on interaction with the user interface by the healthcare facilitator during a facilitator intervention sequence of the engagement session, the community health application controlling the user interface by presenting displays on the display device based on the selected facilitator intervention and reading data from the user interface in relation to the displays, the healthcare facilitator performing the interventions identified in the displays and providing comments and feedback data regarding such interventions in corresponding fields of the displays.

9. The method of claim 4, further comprising:
providing the facilitator referral selected by the community health application to the community health client based on interaction with the user interface by the healthcare facilitator during a facilitator referral sequence of the engagement session, the community health application controlling the user interface by presenting displays on the display device based on the selected facilitator referral and reading data from the user interface in relation to the displays, the healthcare facilitator performing the referrals identified in the displays and providing comments and feedback data regarding such referrals in corresponding fields of the displays.

10. The method of claim 9, wherein the providing of the facilitator referral includes providing an on-site referral to the community health client, the method further comprising:
sending an electronic message to an on-site healthcare provider to provide at least one of notice of the on-site referral to the on-site healthcare provider, at least a portion of the first and second client information from the engagement session with the community health client to the on-site healthcare provider, and at least a recommendation of a schedule for a referral session with the community health client to the on-site healthcare provider.

11. The method of claim 9, wherein the providing of the facilitator referral includes providing an off-site referral to the community health client, the method further comprising:
sending an electronic message to an off-site healthcare provider to provide at least one of notice of the off-site referral to the off-site healthcare provider, at least a portion of the first and second client information from the engagement session with the community health client to the off-site healthcare provider, and at least a recommendation of a schedule for a referral session with the community health client to the off-site healthcare provider.

12. The method of claim 4, further comprising:
at least temporarily storing the first and second client information from the engagement session with the community health client in a storage device at the computing device;
transmitting the first and second client information from the engagement session with the community health client from the storage device at the computing device to a centralized community health information database of a server computing system, the server computing system being accessible to the computing device; and
storing the first and second client information from the engagement session with the community health client in the centralized community health information database of the server computing system with other client information from other engagement sessions with the community health client.

13. A non-transitory computer-readable medium storing program instructions that, when executed by at least one processor, cause a computerized system to perform a method for improving healthcare through community engagement, the method comprising the method of claim 1.

14. A method for improving healthcare through community engagement, comprising:
storing client information from engagement sessions with a plurality of community health clients in a centralized community health information database of a server computing system, the client information from the engagement sessions being captured electronically during the engagement sessions by healthcare facilitators using a community health application on computing devices, each engagement session including a client identification sequence controlled by the community health application to identify the corresponding community health client, collect the client information from the community health client, and select a clinical sequence for the engagement session based on the client information;
providing an authorized person with access to the client information in the centralized community health information database via a remote computing device with access to the server computing system;
processing a database query from the remote computing device at the server computing system; and
providing results of the database query from the server computing system to the remote computing device.

15. The method of claim 14, further comprising:
at least temporarily storing a snapshot of the centralized community health information database at the server computing system;
providing an authorized hospital electronic medical record (EMR)/electronic health record (EHR) system with access to the snapshot of the centralized community health information database via a secure data transfer gateway and an application program interface (API) to the server computing system;
processing a batch download instruction file from the hospital EMR/EHR system at the server computing system; and
downloading at least a portion of client information from the server computing system to the hospital EMR/EHR system via the API and the secure data transfer gateway in response to the batch download instruction file.

16. The method of claim 14, further comprising:
at least temporarily storing a snapshot of at least a portion of the centralized community health information database at the server computing system;
processing the client information in the snapshot to de-identify community health clients associated with the client information to clean, transform, and enhance the client information to form anonymous community health information compatible with a data warehouse stored in a cloud-based computing system; and
uploading the anonymous community health information from the server computing system to the data warehouse stored in the cloud-based computing system via a secure data transfer gateway.

17. The method of claim 16, comprising:
storing the anonymous community health information from the engagement sessions with the plurality of community health clients in the data warehouse of the cloud-based computing system;
providing a second authorized person with access to the anonymous community health information in the data warehouse via a second remote computing device with access to the cloud-based computing system;
processing a data warehouse query from the second remote computing device at the data warehouse;
providing results of the data warehouse query from the cloud-based computing system to the second remote computing device.

18. A non-transitory computer-readable medium storing program instructions that, when executed by at least one processor, cause a computerized system to perform a method for improving healthcare through community engagement, the method comprising the method of claim 14.

19. A computerized system for improving healthcare through community engagement, comprising:
a computing device configured for use by a healthcare facilitator for an engagement session with a candidate patient from a community, the computing device including:
at least one processor and associated memory;
a user interface;
a display device; and
at least one storage device;
wherein the at least one processor, associated memory, user interface, display device, and at least one storage device are configured to initialize a community health application on the computing device for the engagement session;
wherein the at least one processor, associated memory, user interface, display device, and at least one storage device are configured to perform a client identification sequence using the community health application to enroll the candidate patient as a new community health client or lookup the candidate patient as an existing community health client;
wherein the at least one processor, associated memory, user interface, display device, and at least one storage device are configured to collect first client information from the community health client via interaction with the user interface of the computing device by the healthcare facilitator during the engagement session, the community health application controlling the user interface by presenting displays on the display device and reading data from the user interface in relation to the displays;
wherein the at least one processor, associated memory, and at least one storage device are configured to process the first client information to select a clinical sequence for the engagement session with the community health client based at least in part on the first client information.

20. The computerized system of claim 19, wherein the at least one processor, associated memory, and at least one storage device are configured to at least temporarily store the first client information from the engagement session with the community health client in the at least one storage device, the computerized system further comprising:
a server computing system configured to store a centralized community health information database; and
the computing device, further comprising:
a network interface device;
wherein the at least one processor, associated memory, at least one storage device, and network interlace device are configured to transmit the first client information from the engagement session with the community health client to the centralized community health information database of the server computing system via the network interface device;
wherein the server computing system is configured to store the first client information from the engagement session with the community health client in the centralized community health information database with other client information from other engagement sessions with the community health client.

21. The computerized system of claim 19, wherein the at least one processor, associated memory, user interface, display device, and at least one storage device are configured to collect second client information from the community health client via interaction with the user interface by the healthcare facilitator during the selected clinical sequence, the community health application controlling the user interface by presenting displays on the display device based on the selected clinical sequence and reading data from the user interface in relation to the displays;
wherein the at least one processor, associated memory, and at least one storage device are configured to process the second client information to select at least one of a facilitator intervention and a facilitator referral for the community health client based at least in part on the second client information.

22. The computerized system of claim 21, wherein the at least one processor, associated memory, user interface, display device, and at least one storage device are configured to collect supplemental client information from the community health client via interaction with the user interface by the healthcare facilitator during a supplemental clinical sequence of the engagement session, the community health application controlling the user interface by presenting displays on the display device based on the supplemental client information needed and reading data from the user interface in relation to the displays;
wherein the at least one processor, associated memory, and at least one storage device are configured to process the second client information and supplemental client information to select at least one of a facilitator intervention and a facilitator referral for the community health client based at least in part on the second and supplemental client information.

23. A computerized system for improving healthcare through community engagement, comprising:
a server computing system, including:
at least one processor and associated memory;
at least one storage device configured to store a centralized community health information database; and
at least one network interface device;
wherein the at least one processor and associated memory and the at least one storage device are configured to store client information from engagement sessions with a plurality of community health clients in the centralized community health information database, the client information from the engagement sessions being captured electronically during the engagement sessions by healthcare facilitators using a community health application on computing devices, each engagement session including a client identification sequence controlled by the community health application to identify the corresponding community health client, collect the client information from the community health client, and select a clinical sequence for the engagement session based on the client information, the computing devices being configured to transmit the client information from the engagement sessions to the server computing system and the server computing system being configured to receive the client information via the at least one network interface device;
wherein the at least one processor and associated memory, the at least one storage device, and the at least one network interface device are configured to provide an authorized person with access to the client information in the centralized community health information database via a remote computing device;
wherein the at least one processor and associated memory and the at least one storage device are configured to process a database query from the remote computing device at the server computing system;
wherein the at least one processor and associated memory, the at least one storage device, and the at least one network interface device are configured to provide results of the database query from the server computing system to the remote computing device.

24. The computerized system of claim 23, wherein the at least one processor and associated memory and the at least one storage device are configured to at least temporarily store a snapshot of the centralized community health information database at the server computing system;
wherein the at least one processor and associated memory, the at least one storage device, and the at least one network interface device are configured to provide an authorized hospital electronic medical record (EMR)/electronic health record (EHR) system with access to the snapshot of the centralized community health information database via a secure data transfer gateway and an application program interface (API) to the server computing system;
wherein the at least one processor and associated memory and the at least one storage device are configured to process a batch download instruction file from the hospital EMR/EHR system at the server computing system;
wherein the at least one processor and associated memory, the at least one storage device, and the at least one network interface device are configured to download at least a portion of client information from the server computing system to the hospital EMR/EHR system via the API and the secure data transfer gateway in response to the batch download instruction file.

25. The computerized system of claim 23, wherein the at least one processor and associated memory and the at least one storage device are configured to at least temporarily store a snapshot of at least a portion of the centralized community health information database at the server computing system;
wherein the at least one processor and associated memory and the at least one storage device are configured to process the client information in the snapshot to de-identify community health clients associated with the client information to clean, transform, and enhance the client information to form anonymous community health information compatible with a data warehouse stored in a cloud-based computing system;
wherein the at least one processor and associated memory, the at least one storage device, and the at least one network interface device are configured to upload the anonymous community health information from the server computing system to the data warehouse stored in the cloud-based computing system via a secure data transfer gateway.

26. The computerized system of claim 25, wherein the data warehouse of the cloud-based computing system is configured to store the anonymous community health information from the engagement sessions with the plurality of community health clients;
wherein the data warehouse of the cloud-based computing system is configured to provide a second authorized person with access to the anonymous community health information in the data warehouse via a second remote computing device with access to the cloud-based computing system;
wherein the data warehouse of the cloud-based computing system is configured to process a data warehouse query from the second remote computing device;
wherein the data warehouse of the cloud-based computing system is configured to provide results of the data warehouse query to the second remote computing device.
